(19) United States
(12) Patent Application Publication
(10) Pub. No.: US 2023/0172969 A1
(43) Pub. Date: Jun. 8, 2023

(54) Ophthalmic Composition

(71) Applicant: Rohto Pharmaceutical Co., Ltd., Osaka-shi, Osaka (JP)

(72) Inventors: Tatsuya Ando, Osaka-shi, Osaka (JP); Yoko Mitsuguchi, Osaka-shi, Osaka (JP); Sachiko Matsumoto, Osaka-shi, Osaka (JP); Atsuko Nakata, Osaka-shi, Osaka (JP)

(73) Assignee: Rohto Pharmaceutical Co., Ltd., Osaka-shi, Osaka (JP)

(21) Appl. No.: 17/921,065

(22) PCT Filed: Mar. 31, 2021
(86) PCT No.: PCT/JP2021/014059

§ 371 Date: Oct. 24, 2022

(30) Foreign Application Priority Data
Apr. 30, 2020 (JP) 2020-079971

(51) Int. Cl. A61K 31/737; A61K 9/00; A61K 31/23; A61K 31/355; A61K 31/205; A61K 31/198; A61K 31/27; A61K 47/38; A61K 31/4415; A61K 31/16; A61P 27/04; A61K 31/4166; A61K 31/704

(52) U.S. Cl. CPC A61K 31/737; A61K 9/0048; A61K 31/23; A61K 31/355; A61K 31/205; A61K 31/198; A61K 31/27; A61K 47/38; A61K 31/4415; A61K 31/16; A61P 27/04; A61K 31/4166; A61K 31/704

(57) ABSTRACT

The present invention relates to an ophthalmic composition for suppressing eye dryness, comprising at least one selected from the group consisting of chondroitin sulfate having a weight average molecular weight of 30,000 to 50,000 and a salt thereof.

### TECHNICAL FIELD

The present invention relates to an ophthalmic composition.

### BACKGROUND ART

Eye dryness (dry eye) occurs due to a cause such as air dryness due to the use of air conditioners, a decrease in the number of blinks due to long-hour personal computer work, or wearing of contact lenses, and is associated with various unpleasant symptoms such as feeling of ocular pain. Although the symptoms of eye dryness may be relaxed by, for example, applying artificial lachrymal fluid frequently, almost nothing is known about ophthalmic preparations for preventing eye dryness beforehand.

Chondroitin sulfate or a salt thereof is a type of acidic mucopolysaccharide, and is contained in an ophthalmic preparation for promoting energy metabolism, relieving eyestrain by promoting metabolism or cellular respiration, resupplying lachrymal fluid components, and the like (for example, Patent Literature 1).

### CITATION LIST

### Patent Literature

Patent Literature 1
Japanese Unexamined Patent Publication No. 2011-148791

### SUMMARY OF INVENTION

### Solution Problem

An object of the present invention is to provide an ophthalmic composition that can suppress eye dryness.

### Solution to Problem

The present inventors have surprisingly found that sodium chondroitin sulfate having a specific weight average molecular weight suppresses corneal disorder due to eye dryness remarkably, and also suppresses contact lens dryness thanks to its high affinity to contact lenses.

For example, the present invention provides the following inventions.
[1] An ophthalmic composition for suppressing eye dryness, comprising at least one selected from the group consisting of chondroitin sulfate having a weight average molecular weight of 30,000 to 50,000 and a salt thereof.
[2] An ophthalmic composition for suppressing contact lens dryness, comprising at least one selected from the group consisting of chondroitin sulfate having a weight average molecular weight of 30,000 to 50,000 and a salt thereof.
[3] The ophthalmic composition according to [1] or [2], further comprising at least one selected from the group consisting of an anti-inflammatory agent, vitamin A, vitamin B, vitamin E, aminoethylsulfonic acid and a salt thereof, aspartic acid and a salt thereof, neostigmine and a salt thereof, and a cellulosic polymer compound.

For example, the present invention provides the following inventions.
[2-1] An ophthalmic composition, comprising at least one selected from the group consisting of chondroitin sulfate having a weight average molecular weight of 30,000 to 50,000 and a salt thereof and at least one selected from the group consisting of vitamin A, and aminoethylsulfonic acid and a salt thereof
[2-2] An ophthalmic composition, comprising at least one selected from the group consisting of chondroitin sulfate having a weight average molecular weight of 30,000 to 50,000 and a salt thereof and at least one selected from the group consisting of an anti-inflammatory agent, vitamin A, vitamin B, vitamin E, aminoethylsulfonic acid and a salt thereof, aspartic acid and a salt thereof, neostigmine and a salt thereof, and a cellulosic polymer compound.

### Advantageous Effects of Invention

According to the present invention, an ophthalmic composition that can suppress eye dryness can be provided. According to the present invention, an ophthalmic composition that can suppress contact lens dryness can be provided.

### BRIEF DESCRIPTION OF DRAWING

FIG. 1 is a graph showing the results of Test Example 1.

### DESCRIPTION OF EMBODIMENTS

Hereinafter, aspects for implementing the present invention will be described in detail. However, the present invention is not limited to the following embodiments.

The unit “%” for content herein means “% w/v” unless otherwise specified, and is synonymous with “g/100 mL”.

The ophthalmic composition according to the present embodiment contains at least one selected from the group consisting of chondroitin sulfate having a weight average molecular weight of 30,000 to 50,000 and a salt thereof (also described merely as an “(A) component”).

Chondroitin sulfate and the salt thereof are not particularly limited as long as they are pharmacologically (pharmaceutically) or physiologically acceptable medicinally.

Examples of the salt of chondroitin sulfate include alkali metal salts and alkaline-earth metal salts. Examples of the alkali metal salts include a sodium salt and a potassium salt. Examples of the alkaline-earth metal salts include a magnesium salt and a calcium salt.

As chondroitin sulfate and the salt thereof, chondroitin sulfate and an alkali metal salt thereof are preferable, chondroitin sulfate and sodium chondroitin sulfate are more preferable, and sodium chondroitin sulfate is further preferable.

Although chondroitin sulfate and the salt thereof may be natural or synthetic, chondroitin sulfate and the salt thereof derived from animals (preferably mammals, fish, mollusks, and the like; more preferably bovines, sharks, squids, and the like; and further preferably sharks), which are natural, are appropriately used commonly, and chondroitin sulfate derived especially from sharks is appropriately used.

Commercial products can be used as chondroitin sulfate and the salt thereof. Chondroitin sulfate and the salt thereof may be used singly or in combinations of two or more thereof.

The “weight average molecular weight” herein means a value calculated by the calculation expression described below based on the intrinsic viscosity of chondroitin sulfate and the salt thereof (Biol. Rev (1967), 42, 499-551). The intrinsic viscosity can be measured by the viscometry described in general test methods in the Japanese Pharmacopeia, 17th edition. As long as the weight average molecular weight of chondroitin sulfate and the salt thereof calculated by the above-mentioned method is in the range of 30,000 to 50,000, the weight average molecular weight is not particularly limited, it is however preferable that the weight average molecular weight be 31,000 to 49,000, and it is more preferable that the weight average molecular weight be 32,000 to 48,000.

### <Calculation Expression of Weight Average Molecular Weight>

In the case of chondroitin sulfate C:

[η]=5.8×10⁻⁴M^{-7.4}

In the case of chondroitin sulfate A or B:

[η]=3.1×10⁻⁴M^{-7.4}

In the above-mentioned expressions, η represents the intrinsic viscosity, and M represents the weight average molecular weight.

The content of the (A) component in the ophthalmic composition according to the present embodiment is not particularly limited, and is suitably set depending on the types and contents of other blended components, the purpose and preparation form of the ophthalmic composition, and the like. The content of the (A) component is, for example, usually 0.001 to 5% w/v, it is preferable that the content be 0.005 to 5% w/v, it is more preferable that the content be 0.008 to 4% w/v, it is further preferable that the content be 0.01 to 3% w/v, it is further more preferable that the content be 0.05 to 2% w/v, it is particularly preferable that the content be 0.1 to 1% w/v, and it is the most preferable that the content be 0.3 to 1% w/v based on the total amount of the ophthalmic composition from the viewpoint of producing the effect of the present invention more remarkably.

The ophthalmic composition according to the present embodiment may further contain at least one selected from the group consisting of an anti-inflammatory agent, vitamin A, vitamin B, vitamin E, aminoethylsulfonic acid and a salt thereof, aspartic acid and a salt thereof, neostigmine and a salt thereof, and a cellulosic polymer compound (also described merely as a “(B) component”). When the ophthalmic composition contains the (B) component, the effect of the present invention is produced more remarkably.

As confirmed in Test Example described below, the ophthalmic composition according to the present embodiment containing the (A) component and the (B) component also exhibits the effect of suppressing liquid remaining in piping at the time of filling a container or feeding liquid, the effect of enhancing preservative effect, the effect of facilitating blinks regardless of the viscosity of the preparation upon application to the eyes (even though the preparation is a highly viscous preparation), the effect of suppressing an uncomfortable feeling at the time of blinks regardless of the viscosity of the preparation upon application to the eyes (even though the preparation is a highly viscous preparation), the effect of suppressing viscosity change due to daylight, the effect of suppressing the deposition of the components (white residues), the effect of suppressing the coloration of the preparation by daylight, the effect of suppressing the coloration of the preparation by ultraviolet rays, the effect of suppressing a change in appearance (transparency) due to heat, the effect of enhancing the cell viability, the effect of suppressing damage to ocular cells by external stimuli (blinks, external stimulus derived from contact lenses (when contact lenses are put in and removed and while contact lenses are worn), rubbing the eyes, foreign body invasion (pollen, air pollutants, eyelashes, foreign bodies related to eye makeup, other foreign bodies, and the like), and the effect of reducing liquid remaining in containers after use.

[Anti-Inflammatory Agent]

The anti-inflammatory agent is a compound having anti-inflammatory action or antiphlogistic action and a salt thereof. The anti-inflammatory agent is not particularly limited as long as it is pharmacologically (pharmaceutically) or physiologically acceptable medicinally.

Specific examples of the anti-inflammatory agent include epsilon-aminocaproic acid, allantoin, berberine, azulenes (azulene, azulene sulfonic acid, chamazulene, guaiazulene, and the like), glycyrrhizinic acid, a zinc salt, lysozyme, lysozyme chloride, celecoxib, rofecoxib, indomethacin, diclofenac, bromfenac, piroxicam, meloxicam, methyl salicylate, glycol salicylate, tranexamic acid, ibuprofen, ibuprofen piconol, bufexamac, butyl flufenamate, bendazac, ketoprofen, felbinac, pranoprofen, and salts thereof. As the anti-inflammatory agent, allantoin, glycyrrhizinic acid and the salt thereof, and the zinc salt are preferable, and allantoin, glycyrrhizinic acid and the salt thereof are more preferable. As the glycyrrhizinic acid and the salt thereof, an alkali metal salt or an ammonium salt of glycyrrhizinic acid is preferable, and dipotassium glycyrrhizinate and monoammonium glycyrrhizinate are more preferable, and dipotassium glycyrrhizinate is further preferable. As the zinc salt, zinc sulfate or zinc lactate is preferable, and zinc sulfate is more preferable. The zinc salt may be a hydrate (for example, zinc sulfate heptahydrate).

Commercial products can be used as the anti-inflammatory agent. The anti-inflammatory agents may be used singly or in combinations of two or more thereof.

When the anti-inflammatory agent is used as the (B) component, the content of the anti-inflammatory agent in the ophthalmic composition according to the present embodiment is not particularly limited, and the content is suitably set depending on the type of the anti-inflammatory agent, the types and contents of other blended components, the purpose and preparation form of the ophthalmic composition, and the like. As to the content of the anti-inflammatory agent, it is preferable that the total content of the anti-inflammatory agent be 0.0001 to 10% w/v, it is more preferable that the total content be 0.001 to 5% w/v, it is further preferable that the total content be 0.005 to 3% w/v, it is further more preferable that the total content be 0.01 to 1% w/v, and it is particularly preferable that the total content be 0.03 to 0.5% w/v based on the total amount of the ophthalmic composition from the viewpoint of producing the effect of the present invention more remarkably. The total content of the anti-inflammatory agent may be 0.25% w/v.

When the anti-inflammatory agent is used as the (B) component, the content ratio of the anti-inflammatory agent to the (A) component in the ophthalmic composition according to the present embodiment is not particularly limited, and the content ratio is suitably set depending on the types of the (A) component and the anti-inflammatory agent, the types and contents of other blended components, the purpose and preparation form of the ophthalmic composition, and the like. For example, as to the content ratio of the anti-inflammatory agent to the (A) component, it is preferable that the total content of the anti-inflammatory agent be 0.0002 to 1000 parts by mass, it is more preferable that the total content be 0.001 to 500 parts by mass, it is further preferable that the total content be 0.01 to 100 parts by mass, it is further more preferable that the total content be 0.05 to 50 parts by mass, it is further more preferable that the total content be 0.05 to 30 parts by mass, and it is particularly preferable that the total content be 0.1 to 10 parts by mass based on the total content of the (A) component contained in the ophthalmic composition according to the present embodiment of 1 part by mass from the viewpoint of enhancing the effect of the present invention further. The total content of the anti-inflammatory agent based on the total content of the (A) component of 1 part by mass may be 0.5 parts by mass.

[Vitamin A]

Vitamin A is not particularly limited as long as it is pharmacologically (pharmaceutically) or physiologically acceptable medicinally. Specific examples of the vitamin A include retinol, retinal, retinoic acid, derivatives thereof, and salts thereof.

Examples of the derivatives of the vitamin A include esters with monovalent carboxylic acids such as retinol palmitate, retinol acetate, retinol butyrate, retinol propionate, retinol octylate, retinol laurylate, retinol oleate, and retinol linolenate.

Examples of the salts of the vitamin A include organic acid salts [for example, monocarboxylates (acetates, trifluoroacetates, butyrates, palmitates, stearates, and the like), polyvalent carboxylates (fumarates, maleates, succinates, malonates, and the like), oxycarboxylates (lactates, tartrates, citrates, and the like), organic sulfonates (methanesulfonates, toluenesulfonates, tosylates, and the like), and the like], inorganic acid salts (for example, hydrochlorides, sulfates, nitrates, hydrobromates, phosphates, and the like), salts with organic bases (salts with organic amines such as methylamine, triethylamine, triethanolamine, morpholine, piperazine, pyrrolidine, tripyridine, picoline; and the like), and salts with inorganic bases [for example, ammonium salts; salts with metals such as alkali metals (sodium, potassium, and the like), alkaline-earth metals (calcium, magnesium, and the like), aluminum; and the like].

As the vitamin A, derivatives of retinol are preferable, and esters of retinol and monovalent carboxylic acids are more preferable, and retinol palmitate and retinol acetate are further preferable, and retinol palmitate is further more preferable.

As the vitamin A, synthetic products may be used, or extracts obtained from natural products (for example, vitamin A oil, and the like) may be used. The vitamin A oil is fatty oil obtained from animal tissue containing retinol, and the like, a concentrate thereof, or a mixture obtained by adding vegetable oil thereto optionally. Commercial products may be used as the vitamin A. The vitamin A may be used singly or in combinations of two or more thereof.

It is preferable that the total content of the vitamin A in the ophthalmic composition according to the present embodiment be 1,000 to 300,000 IU/100 mL, it is more preferable that the total content be 5,000 to 300,000 IU/100 mL, it is further preferable that the total content be 10,000 to 100,000 IU/100 mL, it is further more preferable that the total content be 30,000 to 55,000 IU/100 mL, it is more further preferable that the total content be 35,000 to 55,000 IU/100 mL, and it is particularly preferable that the total content be 45,000 to 55,000 IU/100 mL based on the total amount of the ophthalmic composition.

“IU” means an international unit determined by a technique described in the vitamin A assay and the like in the Japanese Pharmacopeia, 17th edition. In drug provisions of the Japanese Pharmacopeia, 17th edition, it is stated, for example, that retinol acetate contains 2,500,000 units or more of vitamin A per 1 g of retinol acetate, and retinol palmitate contains 1,500,000 units or more of vitamin A per 1 g of retinol palmitate.

When the vitamin A is used as the (B) component, the content ratio of the vitamin A to the (A) component in the ophthalmic composition according to the present embodiment is not particularly limited, and is suitably set depending on the types of the (A) component and the vitamin A, the types and contents of other blended components, the purpose and preparation form of the ophthalmic composition, and the like. As to the content ratio of the vitamin A to the component (A), for example, it is preferable that the total content of the vitamin A be 1,000 to 3,000,000 IU/g, it is more preferable that the total content be 10,000 to 1,000,000 IU/g, it is further preferable that the total content be 35,000 to 550,000 IU/g, and it is further more preferable that the total content be 45,000 to 550,000 IU/g based on the total content of the (A) component contained in the ophthalmic composition according to the present embodiment of 1 part by mass from the viewpoint of enhancing the effect of the present invention further.

[Vitamin B]

Vitamin B is not particularly limited as long as it is pharmacologically (pharmaceutically) or physiologically acceptable medicinally.

Specific examples of the vitamin B include flavin adenine dinucleotide and a salt thereof (flavin adenine dinucleotide sodium and the like), cobalamins (cyanocobalamin, methylcobalamin, and the like), pantothenic acid and a salt thereof (for example, sodium pantothenate, potassium pantothenate, calcium pantothenate, magnesium pantothenate, and the like), panthenol, pyridoxine or a salt thereof (pyridoxine hydrochloride and the like), pyridoxal and a salt thereof (pyridoxal phosphate and the like). As the vitamin B, panthenol, pyridoxine or the salt thereof is preferable.

As the vitamin B, commercial products can also be used. The vitamin B may be use singly or in combinations of two or more thereof.

When the vitamin B is used as the component (B), the content of the vitamin B in the ophthalmic composition according to the present embodiment is not particularly limited, and the content is suitably set depending on the type of the vitamin B, the types and the contents of other blended components, the purpose and preparation form of the ophthalmic composition, and the like. As to the content of the vitamin B, it is preferable that the total content of the vitamin B be 0.0001 to 5% w/v, it is more preferable that the total content be 0.0005 to 1% w/v, it is further preferable that the total content be 0.001 to 1% w/v, it is further more preferable that the total content be 0.005 to 0.5% w/v, and it is particularly preferable that the total content be 0.01 to 0.1% w/v based on the total amount of the ophthalmic composition from the viewpoint of producing the effect of the present invention more remarkably.

When the vitamin B is used as the (B) component, the content ratio of the vitamin B to the (A) component in the ophthalmic composition according to the present embodiment is not particularly limited, and is suitably set depending on the types of the (A) component and the vitamin B, the types and contents of other blended components, the purpose and preparation form of the ophthalmic composition, and the like. As to the content ratio of the vitamin B to the (A) component, for example, it is preferable that the total content of the vitamin B be 0.00002 to 1000 parts by mass, it is more preferable that the total content be 0.0001 to 500 parts by mass, it is more preferable that the total content be 0.001 to 100 parts by mass, it is further preferable that the total content be 0.005 to 50 parts by mass, it is further more preferable that the total content be 0.01 to 30 parts by mass, and it is particularly preferable that the total content be 0.05 to 1 part by mass based on the total content of the (A) component contained in the ophthalmic composition according to the present embodiment of 1 part by mass from the viewpoint of enhancing the effect of the present invention further.

[Vitamin E]

Vitamin E is not particularly limited as long as it is pharmacologically (pharmaceutically) or physiologically acceptable medicinally. Specific examples of the vitamin E include tocopherol, tocotrienol, derivatives thereof, and salts thereof. Tocopherol and tocotrienol may be any of an α-isomer, a β-isomer, a γ-isomer, and a δ-isomer, and may be any of a d-isomer and a dl-isomer.

Examples of the derivatives of the vitamin E include esters with organic acids such as tocopherol acetate, tocopherol succinate, tocopherol nicotinate, and tocopherol linolenate.

Examples of the salts of vitamin E include organic acid salts (lactates, acetates, butyrates, trifluoroacetates, fumarates, maleates, tartrates, citrates, succinates, malonates, methanesulfonates, toluenesulfonates, tosylates, palmitates, stearates, and the like), inorganic acid salts (for example, hydrochlorides, sulfates, nitrates, hydrobromates, phosphates, and the like), salts with organic bases (salts with organic amines such as methylamine, triethylamine, triethanolamine, morpholine, piperazine, pyrrolidine, amino acids, tripyridine, and picoline, and the like), and salts with inorganic bases (for example, ammonium salts; salts with metals such as alkali metals such as sodium and potassium, alkaline-earth metals such as calcium and magnesium, and aluminum; and the like).

As the vitamin E, d-α-tocopherol, dl-α-tocopherol, β-tocopherol, γ-tocopherol, δ-tocopherol, vitamin E acetate (for example, tocopherol acetate), vitamin E nicotinate, vitamin E succinate, and vitamin E linolenate are preferable, and tocopherol acetate (for example, d-α-tocopherol acetate, dl-α-tocopherol acetate, and the like) is more preferable.

The vitamin E may be either natural or synthetic. Commercial products can also be used as the vitamin E. The vitamin E may be used singly or in combinations of two or more thereof.

As to the content of the vitamin E in the ophthalmic composition according to the present embodiment, it is preferable that the total content of the vitamin E be 0.0001 to 0.5% w/v, it is more preferable that the total content be 0.001 to 0.1% w/v, it is further preferable that the total content be 0.005 to 0.05% w/v, and it is further more preferable that the total content be 0.01 to 0.05% w/v based on the total amount of the ophthalmic composition.

When the vitamin E is used as the (B) component, the content ratio of the vitamin E to the (A) component in the ophthalmic composition according to the present embodiment is not particularly limited, and the content ratio is suitably set depending on the types of the (A) component and the vitamin E, the types and contents of other blended components, the purpose and preparation form of the ophthalmic composition, and the like. As to the content ratio of the vitamin E to the component (A), for example, it is preferable that the total content of the vitamin E be 0.0001 to 50 parts by mass, it is more preferable that the total content be 0.001 to 20 parts by mass, it is further preferable that the total content be 0.005 to 10 parts by mass, it is further more preferable that the total content be 0.01 to 5 parts by mass, and it is particularly preferable that the total content be 0.01 to 0.5 parts by mass based on the total content of the (A) component contained in the ophthalmic composition according to the present embodiment of 1 part by mass from the viewpoint of enhancing the effect of the present invention further.

[Aminoethylsulfonic Acid and Salt Thereof]

Aminoethylsulfonic acid (taurine) and a salt thereof are not particularly limited as long as they are pharmacologically (pharmaceutically) or physiologically acceptable medicinally.

Examples of the salt of the aminoethylsulfonic acid include salts with organic bases (salts with organic amines such as methylamine, triethylamine, triethanolamine, morpholine, piperazine, pyrrolidine, tripyridine, and picoline) and salts with inorganic bases [for example, ammonium salts and salts with metals such as alkali metal (sodium, potassium, and the like), alkaline-earth metals (calcium, magnesium, and the like), aluminum].

As aminoethylsulfonic acid and the salt thereof, aminoethylsulfonic acid is preferable.

Commercial products can also be used as aminoethylsulfonic acid and the salt thereof. Aminoethylsulfonic acid and the salt thereof may be used singly or in combinations of two or more thereof.

As to the content of aminoethylsulfonic acid the salt thereof in the ophthalmic composition according to the present embodiment, it is preferable that the total content of aminoethylsulfonic acid and the salt be 0.001 to 10% w/v, it is more preferable that the total content be 0.01 to 5% w/v, it is further preferable that the total content be 0.05 to 3% w/v, and it is further more preferable that the total content be 0.1 to 2% w/v based on the total amount of the ophthalmic composition.

When aminoethylsulfonic acid and the salt thereof are used as the component (B), the content ratio of aminoethylsulfonic acid and the salt thereof to the (A) component in the ophthalmic composition according to the present embodiment is not particularly limited, and is suitably set depending on the types of the (A) component and aminoethylsulfonic acid and the salt thereof, the types and contents of other blended components, the purpose and preparation form of the ophthalmic composition, and the like. As to the content ratio aminoethylsulfonic acid to the component (A) and the salt thereof, for example, it is preferable that the total content of aminoethylsulfonic acid and the salt thereof be 0.001 to 1000 parts by mass, it is more preferable that the total content be 0.01 to 200 parts by mass, it is further preferable that the total content be 0.05 to 100 parts by mass, it is further more preferable that the total content be 0.1 to 20 parts by mass, and it is particularly preferable that the total content be 0.1 to 10 parts by mass based on the total content of the (A) component contained in the ophthalmic composition according to the present embodiment of 1 part by mass from the viewpoint of enhancing the effect of the present invention further.

[Aspartic Acid and Salt Thereof]

Aspartic acid is a compound well-known as an acidic amino acid also called 2-aminobutanedioic acid. Aspartic acid and a salt thereof are not particularly limited as long as they are pharmacologically (pharmaceutically) or physiologically acceptable medicinally. Aspartic acid may be any of an L-isomer, a D-isomer, a DL-isomer, and is preferably an L-isomer.

Examples of the salts of aspartic acid include salts with inorganic bases (for example, an ammonium salt and salts with metals such as alkali metals (sodium, potassium, and the like), alkaline-earth metals (calcium, magnesium, and the like), aluminum, and the like) and salts with organic bases (for example, salts with organic amines such as methylamine, triethylamine, triethanolamine, morpholine, piperazine, pyrrolidine, tripyridine, picoline, and the like). As aspartic acid and the salt thereof, the salts of aspartic acid with the inorganic bases are preferable, and the alkali metal salts and the alkaline-earth metal salts of aspartic acid are more preferable, and potassium aspartate, magnesium aspartate, and magnesium potassium aspartate are further preferable.

Commercial products can also be used as aspartic acid and the salt thereof. Aspartic acid and the salt thereof may be used singly or in combinations of two or more thereof.

The content of aspartic acid and the salt thereof in the ophthalmic composition according to the present embodiment is not particularly limited, and is suitably set depending on the types and contents of other blended components, the preparation form, and the like. As the content of aspartic acid or the salt thereof, for example, it is preferable that the total content of aspartic acid or the salt thereof be 0.001 to 10% w/v, it is more preferable that the total content be 0.01 to 5% w/v, it is further preferable that the total content be 0.05 to 3% w/v, and it is further more preferable that the total content be 0.1 to 2% w/v based on the total amount of the ophthalmic composition.

When aspartic acid and the salt thereof are used as the (B) component, the content ratio of aspartic acid and the salt thereof to the (A) component in the ophthalmic composition according to the present embodiment is not particularly limited, and is suitably set depending on the types of the (A) component and aspartic acid and the salt thereof, the types and contents of other blended components, the purpose and preparation form of the ophthalmic composition, and the like. As to the content ratio of aspartic acid and the salt thereof to the component (A), for example, it is preferable that the total content of aspartic acid and the salt thereof be 0.001 to 1000 parts by mass, it is more preferable that the total content be 0.01 to 300 parts by mass, it is further preferable that the total content be 0.05 to 200 parts by mass, it is further more preferable that the total content be 0.1 to 50 parts by mass, and it is particularly preferable that the total content be 0.1 to 20 parts by mas based on the total content of the (A) component contained in the ophthalmic composition according to the present embodiment of 1 part by mass from the viewpoint of enhancing the effect of the present invention further.

[Neostigmine and Salt Thereof]

Neostigmine and a salt thereof are not particularly limited as long as they are pharmacologically (pharmaceutically) or physiologically acceptable medicinally. Examples of the salt of neostigmine include neostigmine methylsulfate. As neostigmine and the salt thereof, neostigmine methylsulfate is preferable.

Commercial products can also be used as neostigmine and the salt thereof. Neostigmine and the salt thereof may be used singly or in combinations of two or more thereof.

As to the content of neostigmine and the salt thereof in the ophthalmic composition according to the present embodiment, it is preferable that the total content of neostigmine and the salt thereof be 0.0001 to 0.05% w/v, it is more preferable that the total content be 0.0005 to 0.01% w/v, it is further preferable that the total content be 0.0008 to 0.008% w/v, and it is further preferable that the total content be 0.001 to 0.005% w/v based on the total amount of the ophthalmic composition.

When neostigmine and the salt thereof are used as the component (B), the content ratio of neostigmine and the salt thereof to the (A) component in the ophthalmic composition according to the present embodiment is not particularly limited, and is suitably set depending on the types of the (A) component and neostigmine and the salt thereof, the types and contents of other blended components, the purpose and preparation form of the ophthalmic composition, and the like. As to the content ratio of neostigmine and the salt thereof to the component (A), for example, it is preferable that the total content of neostigmine and the salt thereof be 0.0001 to 5 parts by mass, it is more preferable that the total content be 0.0001 to 1 part by mass, it is further preferable that the total content be 0.0005 to 0.8 parts by mass, it is further more preferable that the total content be 0.001 to 0.5 parts by mass, it is particularly preferable that the total content be 0.001 to 0.05 parts by mass based on the total content of the (A) component contained in the ophthalmic composition according to the present embodiment of 1 part by mass from the viewpoint of enhancing the effect of the present invention further.

[Cellulosic Polymer Compound]

The cellulosic polymer compound is not particularly limited as long is it is pharmacologically (pharmaceutically) or physiologically acceptable medicinally.

Examples of the cellulosic polymer compound include methyl cellulose, ethyl cellulose, hydroxyethyl cellulose, hydroxymethyl cellulose, hydroxypropyl cellulose, hydroxypropyl methylcellulose (hypromellose), carboxymethyl cellulose, carboxyethyl cellulose, and salts thereof. As the cellulosic polymer compound, hydroxyethyl cellulose, hydroxypropyl methylcellulose, and salts thereof are preferable, and hydroxypropyl methylcellulose and a salt thereof are more preferable. Examples of such salts include salts with organic bases (basic ammonium salts such as amine salts and arginine, and the like) and salts with inorganic bases (ammonium salts, alkali metal salts such as sodium salts and potassium salts, alkaline-earth metal salts such as calcium salts and magnesium salts, aluminum salts, and the like), and especially the sodium salts, the potassium salts, the calcium salts are more preferable, and the sodium salts are particularly preferable.

As the cellulosic polymer compound, a commercial product can also be used. The cellulosic polymer compounds may be used singly or in combinations of two or more thereof.

When the cellulosic polymer compound is used as the (B) component, the content of the cellulosic polymer compound in the ophthalmic composition according to the present embodiment is not particularly limited, and is suitably set depending on the type of the cellulosic polymer compound, the types and contents of other blended components, the purpose and preparation form of the ophthalmic composition, and the like. As to the content of the cellulosic polymer compound, it is preferable that the total content of the cellulosic polymer compound be 0.0001 to 10% w/v, it is more preferable that the total content be 0.001 to 5% w/v, it is further preferable that the total content be 0.005 to 3% w/v, and it is further more preferable that the total content be 0.01 to 2% w/v based on the total amount of the ophthalmic composition from the viewpoint of producing the effect of the present invention more remarkably.

When the cellulosic polymer compound is used as the (B) component, the content ratio of the cellulosic polymer compound to the (A) component in the ophthalmic composition according to the present embodiment is not particularly limited, and is suitably set depending on the types of the (A) component and the cellulosic polymer compound, the types and contents of other blended components, the purpose and preparation form of the ophthalmic composition, and the like. As to the content ratio of the cellulosic polymer compound to the component (A), for example, it is preferable that the total content of the cellulosic polymer compound be 0.0001 to 500 parts by mass, it is more preferable that the total content be 0.001 to 100 parts by mass, it is further preferable that the total content be 0.005 to 50 parts by mass, and it is further more preferable that the total content be 0.01 to 30 parts by mass based on the total content of the (A) component contained in the ophthalmic composition according to the present embodiment of 1 part by mass from the viewpoint of enhancing the effect of the present invention further.

The ophthalmic composition according to the present embodiment may further contain a (C) surfactant (also referred to as a “(C) component”). When the ophthalmic composition further contains the (C) component, the effect of the present invention is produced more remarkably. The surfactant is not particularly limited as long as it is pharmacologically (pharmaceutically) or physiologically acceptable medicinally, and the surfactant may be any of a nonionic surfactant, an amphoteric surfactant, an anionic surfactant, and a cationic surfactant.

Examples of the nonionic surfactant include POE sorbitan fatty acid esters such as POE(20) sorbitan monolaurate (polysorbate 20), POE(20) sorbitan monopalmitate (polysorbate 40), POE(20) sorbitan monostearate (polysorbate 60), POE(20) sorbitan tristearate (polysorbate 65), and POE(20) sorbitan monooleate (polysorbate 80); POE hydrogenated castor oil such as POE(40) hydrogenated castor oil (polyoxyethylene hydrogenated castor oil 40) and POE(60) hydrogenated castor oil (polyoxyethylene hydrogenated castor oil 60); POE castor oil such as POE(3) castor oil (polyoxyethylene castor oil 3), POE(10) castor oil (polyoxyethylene castor oil 10), and POE(35) castor oil (polyoxyethylene castor oil 35); POE alkyl ether such as POE(9) lauryl ether; POE-POP alkyl ether such as POE(20) POP(4) cetyl ether; and polyoxyethylene-polyoxypropylene block copolymers such as POE(196) POP(67) glycol (poloxamer 407, Pluronic F127) and POE(200) POP(70) glycol. In the compounds exemplified above, the POE represents polyoxyethylene, POP represents polyoxypropylene, and the numbers in and the parentheses represent the numbers of added moles.

Examples of the amphoteric surfactant include alkyldiaminoethylglycines or a salt thereof (for example, a hydrochloride, and the like).

Examples of the anionic surfactant include alkylbenzene sulfonates, alkyl sulfates, polyoxyethylene alkyl sulfates, aliphatic α-sulfomethyl ester, and α-olefin sulfonic acids.

Examples of the cationic surfactant include cetylpyridinium chloride, benzalkonium chloride, and benzethonium chloride.

Among these surfactants, the nonionic surfactant is preferable, and the POE sorbitan fatty acid esters, POE hydrogenated castor oil, the POE-POP block copolymers are more preferable. Commercial products can also be used as the surfactant. The surfactants may be used singly or in combinations of two or more thereof.

The content of the (C) component in the ophthalmic composition according to the present embodiment is not particularly limited, and is suitably set depending on the type of the (C) component, the purpose and preparation form of the ophthalmic composition, and the like. As the content of the component (C), for example, it is preferable that the total content of the component (C) be 0.001 to 3% w/v, it is more preferable that the total content be 0.005 to 2% w/v, it is further preferable that the total content be 0.01 to 1% w/v, and it is particularly preferable that the total content be 0.05 to 1% w/v based on the total amount of the ophthalmic composition from the viewpoint of producing the effect of the present invention more remarkably.

The content ratio of the (C) component to the (A) component in the ophthalmic composition according to the present embodiment is not particularly limited, and is suitably set depending on the types of the (A) component and the (C) component, the types and contents of other blended components, the purpose and preparation form of the ophthalmic composition, and the like. As the content ratio of the (C) component to the (A) component, for example, it is more preferable that the total content of the (C) component be 0.001 parts by mass to 30 parts by mass, it is more preferable that the total content be 0.005 to 20 parts by mass, it is further preferable that the total content be 0.01 to 10 parts by mass, and it is particularly preferable that the total content be 1 to 10 parts by mass based on the total content of the (A) component contained in the ophthalmic composition according to the present embodiment of 1 part by mass from the viewpoint of enhancing the effect of the present invention further.

It is preferable that the ophthalmic composition according to the present embodiment further contain a (D) buffer (also referred to as a “(D) component”). When the ophthalmic composition further contains the (D) component, the effect of the present invention is produced more remarkably. The buffer is not particularly limited as long as it is pharmacologically (pharmaceutically) or physiologically acceptable medicinally. Examples of the buffer include inorganic buffers that are buffers derived from inorganic acids and organic buffers that is buffers derived from organic acids or organic bases.

Examples of the inorganic buffers include borate buffers, phosphate buffers, and carbonate buffers. Examples of the borate buffers include boric acid or salts thereof (alkali metal borates, alkaline-earth metal salt borates, and the like). Examples of the phosphate buffers include phosphoric acid or salts thereof (alkali metal phosphates, alkaline-earth metal phosphates, and the like). Examples of the carbonate buffers include carbonic acid or salts thereof (alkali metal carbonates, alkaline-earth metal carbonates, and the like). As a borate buffer, a phosphate buffer, or a carbonate buffer, a hydrate of the borate, the phosphate or the carbonate may be used. As more specific examples, as the borate buffers, boric acid or a salt thereof (sodium borate, potassium tetraborate, potassium metaborate, ammonium borate, borax, or the like) can be exemplified, as the phosphate buffer, phosphoric acid or a salt thereof (disodium hydrogen phosphate, sodium dihydrogen phosphate, potassium dihydrogen phosphate, trisodium phosphate, tripotassium phosphate, calcium monohydrogen phosphate, calcium dihydrogen phosphate, or the like) can be exemplified, and as the carbonate buffer, carbonic acid or a salt thereof (sodium hydrogen carbonate, sodium carbonate, ammonium carbonate, potassium carbonate, calcium carbonate, potassium hydrogen carbonate, magnesium carbonate, or the like), or the like can be exemplified.

Examples of the organic buffers include citrate buffers, acetate buffers, lactate buffers, succinate buffers, tris buffers, AMPD buffers, and the like. Examples of the citrate buffers include citric acid or a salt thereof (an alkali metal salt citrate, an alkaline-earth metal citrate, or the like). Examples of the acetate buffers include acetic acid or a salt thereof (an alkali metal acetate, an alkaline-earth metal acetate, or the like). Examples of the lactate buffers include lactic acid or a salt thereof (an alkali metal lactate, an alkaline-earth metal lactate, or the like). Examples of the succinate buffers include succinic acid or a salt thereof (an alkali metal succinate, or the like). As a citrate buffer, an acetate buffer, a lactate buffer or a succinate buffer, a hydrate of a citrate, an acetate, a lactate, or a succinate may be used. As more specific examples, as the citric acid buffer, citric acid or a salt thereof (sodium citrate, potassium citrate, calcium citrate, sodium dihydrogen citrate, disodium citrate, or the like) is exemplified, as the acetate buffer, acetic acid or a salt (ammonium acetate, sodium acetate, potassium acetate, calcium acetate, or the like) is exemplified, as the lactate buffer, lactic acid or a salt thereof (sodium lactate, potassium lactate, calcium lactate, or the like) is exemplified, and as the succinate buffer, succinic acid or a salt (monosodium succinate, disodium succinate, or the like) is exemplified. Examples of the tris buffers include trometamol or a salt thereof (trometamol hydrochloride, or the like). Examples of the AMPD buffers include 2-amino-2-methyl-1,3-propanediol or a salt thereof.

As the buffer, a borate buffer (for example, a combination of boric acid and borax, or the like), a phosphate buffer (for example, a combination of disodium hydrogen phosphate and sodium dihydrogen phosphate, and the like), and a tris buffer (for example, trometamol) are preferable, and a borate buffer is more preferable, and boric acid and the salt thereof are further preferable, and a combination of boric acid and borax is further more preferable.

Commercial products may be used as the buffer. The buffers may be used singly or in combinations of two or more thereof.

The content of the (D) component in the ophthalmic composition according to the present embodiment is not particularly limited, and is suitably set depending on the type of the (D) component, the types and contents of other blended components, the purpose and preparation form of the ophthalmic composition, and the like. As the content of the component(D), for example, it is preferable that the total content of the component (D) be 0.01 to 10% w/v, it is more preferable that the total content be 0.05 to 5% w/v, and it is further preferable that the total content be 0.1 to 3% w/v based on the total amount of the ophthalmic composition from the viewpoint of producing the effect of the present invention more remarkably.

The content ratio of the (D) component to the (A) component in the ophthalmic composition according to the present embodiment is not particularly limited, and is suitably set depending on the types of the (A) component and the (D) the component, the types and contents of other blended components, the purpose and preparation form of the ophthalmic composition, and the like. As the content ratio of the (D) component to the component (A), for example, it is preferable that total content of the (D) component be 0.01 to 100 parts by mass, it is more preferable that the total content be 0.05 to 50 parts by mass, and it is further preferable that total content be 0.1 to 30 parts by mass based on the total content of the (A) component contained in the ophthalmic composition according to the present embodiment of 1 part by mass from the viewpoint of enhancing the effect of the present invention further.

The pH of the ophthalmic composition according to the present embodiment is not particularly limited as long as it is in a pharmacologically (pharmaceutically) or physiologically acceptable range medicinally. As the pH of the ophthalmic composition according to the present embodiment, for example, the pH may be 4.0 to 9.5, and it is preferable that the pH be 4.0 to 9.0, it is more preferable that the pH be 4.5 to 9.0, it is further preferable that the pH be 4.5 to 8.5, it is further more preferable that the pH be 5.0 to 8.5, it is particularly preferable that the pH be 5.0 to 8.0, it is further particularly preferable that the pH be 5.3 to 7.5, it is the most preferable that the pH be 5.3 to 7.0.

In the ophthalmic composition according to the present embodiment, the osmotic pressure ratio can be adjusted to an osmotic pressure ratio in a range acceptable in the living body when needed. Although the suitable osmotic pressure ratio can be suitably set depending on the purpose, the preparation form, the using method, and the like of the ophthalmic composition, for example, the osmotic pressure ratio can be adjusted to 0.4 to 5.0, it is preferable that the osmotic pressure ratio be adjusted to 0.6 to 3.0, it is more preferable that the osmotic pressure ratio be adjusted to 0.8 to 2.2, and it is further preferable that the osmotic pressure ratio be adjusted to 0.8 to 2.0. The osmotic pressure ratio is defined as the osmotic pressure ratio of a sample to 286 mOsm (osmotic pressure of an aqueous 0.9% w/v sodium chloride solution) based on the Japanese Pharmacopeia, 17th edition, and the osmotic pressure is measured with reference to a method for measuring osmotic pressure (cryoscopy) described in the Japanese Pharmacopeia. Sodium chloride (Japanese Pharmacopeia standard reagent) is dried at 500 to 650° C. for 40 to 50 minutes, cooled in a desiccator (silica gel), and 0.900 g of the sodium chloride is weighed accurately, dissolved in purified water, and diluted to 100 mL accurately to prepare a standard solution for measuring an osmotic pressure ratio (aqueous 0.9% w/v sodium chloride solution), or a commercial standard solution for measuring an osmotic pressure ratio (aqueous 0.9% w/v sodium chloride solution) can be used.

The viscosity of the ophthalmic composition according to the present embodiment is not particularly limited as long as it is in a pharmacologically (pharmaceutically) or physiologically acceptable range medicinally. As the viscosity of the ophthalmic composition according to the present embodiment, for example, it is preferable that the viscosity at 20° C. measured with a rotational viscometer (TV-20 type viscometer, manufactured by Toki Sangyo Co., Ltd., rotor; 1° 34′×R24) be 1 to 10000 m·Pas, it is more preferable that the viscosity be 1 to 8000 m·Pas, it is further preferable that the viscosity be 1 to 1000 m·Pas, it is further more preferable that the viscosity be 1 to 100 m·Pas, it is particularly preferable that the viscosity be 1 to 20 m·Pas, and it is the most preferable that the viscosity be 1.5 to 10 m·Pas.

As long as the effect of the present invention is not deteriorated, the ophthalmic composition according to the present embodiment may contain components selected from various pharmacological active components and physiological active components besides the above-mentioned components in combination in suitable amounts. The components are not particularly limited, and examples include, for example, effective components in ophthalmic drugs described in Yoshido/ippanyo iyakuhin seizo hanbai shonin kijun, 2017-nen ban (Approval standard for manufacturing and marketing of pharmaceuticals requiring guidance/OTC pharmaceuticals, 2017 edition) (supervised by SOCIETY FOR REGULATORY SCIENCE OF MEDICAL PRODUCTS). Specific examples of the components to be used in the ophthalmic drugs include the following components.

Antiallergic drug: for example, sodium cromoglycate, tranilast, pemirolast potassium, acitazanolast, amlexanox, ibudilast, or the like.

Antihistamine: for example, diphenhydramine or a salt thereof (for example, diphenhydramine hydrochloride), iproheptine or a salt thereof (for example, iproheptine hydrochloride), chlorpheniramine or a salt thereof (for example, chlorpheniramine maleate), levocabastine or a salt thereof (for example, levocabastine hydrochloride), ketotifen or a salt thereof (for example, ketotifen fumarate), pemirolast potassium, olopatadine or a salt thereof (for example, olopatadine hydrochloride), or the like

Steroid: for example, fluticasone propionate, fluticasone furancarboxylate, mometasone furancarboxylate, beclometasone dipropionate, flunisolide, or the like.

Decongestant: for example, tetrahydrozoline hydrochloride, tetrahydrozoline nitrate, naphazoline hydrochloride, naphazoline nitrate, epinephrine, epinephrine hydrochloride, ephedrine hydrochloride, phenylephrine hydrochloride, dl-methylephedrine hydrochloride, or the like.

Drugs for modulating ocular muscle: for example, cholinesterase inhibitor having an active center similar to acetylcholine, tropicamide, helenien, atropine sulfate, pilocarpine hydrochloride, or the like.

Vitamins: for example, ascorbic acid, sodium ascorbate, or the like.

Amino acids: for example, L-arginine, glutaminic acid, glycine, alanine, lysin, γ-aminobutyric acid, γ-aminovaleric acid, trimethylglycine, salts thereof, and the like.

Astrictive: for example, flowers of zinc, or the like.

Others: for example, sulfamethoxazole, sulfisoxazole, sulfisomidine, salts thereof, and the like.

As long as the effect of the invention is not deteriorated, various additives are suitably selected according to the usual method depending on the purpose and preparation form thereof, and one may be incorporated into the ophthalmic composition, or two or more may be incorporated into the ophthalmic composition in combination in a suitable amount. Examples of such additives include, for example, various additives described in Japanese Pharmaceutical Excipients Directory 2016 (edited by International Pharmaceutical Excipients Council Japan). Examples of representative components include the following additives.

Carrier: for example, water and aqueous solvents such as water-containing ethanol.

Chelating agent: for example, ethylenediaminediacetic acid (EDDA), ethylenediaminetriacetic acid, ethylenediaminetetraacetic acid (EDTA), N-(2-hydroxyethyl) ethylenediaminetriacetic acid (HEDTA), diethylenetriaminepentaacetic acid (DTPA), and the like.

Base: for example, octyldodecanol, titanium oxide, potassium bromide, plastibase, and the like.

pH regulator: for example, hydrochloric acid, acetic acid, sodium hydroxide, potassium hydroxide, calcium hydroxide, magnesium hydroxide, triethanolamine, monoethanolamine, diisopropanolamine, and the like.

Fragrance or refreshing agent: for example, menthol, menthone, camphor, borneol, geraniol, cineole, citronellol, carvone, anethole, eugenol, limonene, linalool, linalyl acetate, thymol, cymene, terpineol, pinene, camphene, isoborneol, fenchene, nerol, myrcene, myrcenol, linalool acetate, lavandulol, eucalyptus oil, bergamot oil, peppermint oil, cool mint oil, spearmint oil, mint oil, fennel oil, cinnamon oil, rose oil, camphor oil, and the like. These may be any of d-isomers, 1-isomers, and dl-isomers.

Thickener: for example, polyvinyl-based polymer compounds such as polyvinylpyrrolidone and polyvinyl alcohol; a carboxyvinyl polymer; guar gum; hydroxypropyl guar gum; gum arabic; karaya gum; xanthan gum; agar; alginic acid and salts thereof (sodium salt and the like); mucopolysaccharides such as heparin analogs, heparin, heparin sulfate, heparan sulfate, heparinoid, hyaluronic acid and salts thereof (sodium and the like); starch; chitin and derivatives thereof; chitosan and derivatives thereof; carrageenan; monosaccharides such as glucose; and the like.

Stabilizer for example, edetic acid, edetates (disodium edetate, calcium disodium edetate, trisodium edetate, and tetrasodium edetate), sodium formaldehyde sulfoxylate (rongalite), aluminum monostearate, glyceryl monostearate, cyclodextrin, monoethanolamine, dibutylhydroxytoluene, sodium hydrogensulfite, sodium pyrosulfite, and the like.

Antiseptic: for example, quaternary ammonium salts of alkylpolyaminoethylglycine (for example, benzalkonium chloride, benzethonium chloride, and the like), chlorhexidine gluconate, polidronium chloride, zinc chloride, sodium benzoate, ethanol, chlorobutanol, sorbic acid, potassium sorbate, sodium dehydroacetate, methyl para-hydroxybenzoate, ethyl para-hydroxybenzoate, propyl para-hydroxybenzoate, butyl para-hydroxybenzoate, oxyquinoline sulfate, phenethyl alcohol, benzyl alcohol, biguanide compounds (specifically polihexanide hydrochloride (polyhexamethylene biguanide), alexidine, and the like), Glokill (commercial name produced by Rohdia Chimie), and the like.

Isotonizing agent: for example, potassium chloride, calcium chloride, sodium chloride, magnesium chloride, potassium acetate, sodium acetate, sodium hydrogen carbonate, sodium carbonate, sodium thiosulfate, magnesium sulfate, glycerin, propylene glycol, sodium hydrogen sulfite, sodium sulfite, and the like.

Sugar alcohol: for example, xylitol, sorbitol, mannitol, glycerin, and the like. These may be any of d-isomers, 1-isomers, and dl-isomers.

Oils: for example, vegetable oils such as sesame oil, castor oil, soybean oil, and olive oil; animal oils such as squalane; mineral oils such as liquid paraffin and Vaseline; and the like.

It is preferable that the ophthalmic composition according to the present embodiment do not contain at least one selected from the group consisting of geraniol, linalyl acetate, limonene, citral, and linalool in an amount of 0.01% or more, and it is more preferable that the ophthalmic composition do not contain the at least one from the viewpoint that the ophthalmic can produce the effect of the invention remarkably.

When the ophthalmic composition according to the present embodiment contains water, as the water content, for example, it is preferable that the water content be 80% w/v or more and less than 100% w/v, it is more preferable that the water content be 85% w/v or more and 99.5% w/v or less, it is further preferable that the water content be 90% w/v or more and 99.2% w/v or less based on the total amount of the ophthalmic composition from the viewpoint of producing the effect of the present invention more remarkably.

Water to be used for the ophthalmic composition according to the present embodiment only has to be a pharmacologically (pharmaceutically) or physiologically acceptable medicinally. Examples of such water include distilled water, common water, purified water, sterile purified water, and water for injection and distilled water for injection. The definitions thereof are based on the Japanese Pharmacopeia, 17th edition.

A desired amount of the (A) component and, if needed, other components such as the component (B) can be added and mixed to desired concentrations to prepare the ophthalmic composition according to the present embodiment. For example, those components can be dissolved or dispersed in purified water, the pH and osmotic pressure are adjusted to a desired pH and a desired osmotic pressure, and the mixture can be sterilization-treated by filtration sterilization to prepare the ophthalmic composition.

The ophthalmic composition according to the present embodiment can take various preparation forms depending on the object. Examples of the preparation form include solutions, gels, and semi-solid preparations (ointment and the like).

The ophthalmic composition according to the present embodiment can be used for example, as an ophthalmic agent (also referred to as an ophthalmic solution or an ophthalmic drug. The ophthalmic agent includes ophthalmic agents that can be applied while contact lenses are worn.), artificial lachrymal fluid, an eyewash agent (also referred to as an eyewash solution or an eyewash drug. The eyewash agent includes eyewash agents that enables eye washing while contact lenses are worn.), a composition for contact lenses [contact lens wearing solution, a composition for contact lens care (a contact lens disinfectant, a preservative for contact lenses, a cleaner for contact lenses, and a cleaning preservative for contact lenses), contact lens package solution, and the like]. The “contact lenses” include hard contact lenses and soft contact lenses (including both ionic and nonionic contact lenses and including both silicone hydrogel contact lenses and non-silicone hydrogel contact lenses).

Since the ophthalmic composition according to the present embodiment exhibits the effect of suppressing eye dryness, the ophthalmic composition can be appropriately used as an ophthalmic composition for suppressing eye dryness. As one embodiment of the present invention, an ophthalmic composition for suppressing eye dryness containing at least one selected from the group consisting of chondroitin sulfate having a weight average molecular weight of 30,000 to 50,000 and a salt thereof is therefore provided. It is believed that the “effect of suppressing eye dryness” used herein is not due to the action of promoting lacrimation but due to the action of reducing damage to ocular cells due to eye dryness, the action of improving affinity for contact lenses, and the like.

As one embodiment of the present invention, a method for suppressing eye dryness using at least one selected from the group consisting of chondroitin sulfate having a weight average molecular weight of 30,000 to 50,000 and a salt thereof is provided. As one embodiment of the present invention, the use of at least one selected from the group consisting of chondroitin sulfate having a weight average molecular weight of 30,000 to 50,000 and a salt thereof for producing an ophthalmic composition for suppressing eye dryness is furthermore provided.

The ophthalmic composition according to the present embodiment can suppress the dryness of the eyes and contact lenses. Various symptoms due to these symptoms can therefore be resolved. That is, since the ophthalmic composition according to the present embodiment suppresses the dryness of the eyes and contact lenses, symptoms such as friction at the time of palpebration, watery eyes, eyestrain, hyperemia, blurred vision, visual function deterioration, inflammation, an itch, an uncomfortable feeling on the eyes, a feeling of foreign objects, pain, photophobia, ocular discomfort (for example, discomfort when hard contact lenses or soft contact lenses are worn), damage to the eye surfaces done by dryness stress, damage to the eye surfaces done by light and other rays, a heavy feeling of the eyelids (the eyelids are heavy), and the disability to continue looking concentratedly accompanying the dryness of the eyes and contact lenses can be reduced.

Since the ophthalmic composition according to the present embodiment has a high affinity for contact lenses, and exhibits the effect of suppressing contact lens dryness, the ophthalmic composition can be appropriately used as an ophthalmic composition for suppressing contact lens dryness. In this embodiment, it is preferable that the contact lenses be soft contact lenses, and it is more preferable that the contact lenses be silicone hydrogel contact lenses from the viewpoint of producing the effect of the present invention more remarkably.

As one embodiment of the present invention, a method for suppressing contact lens dryness using at least one selected from the group consisting of chondroitin sulfate having a weight average molecular weight of 30,000 to 50,000 and a salt thereof is provided. As one embodiment of the present invention, the use of at least one selected from the group consisting of chondroitin sulfate having a weight average molecular weight of 30,000 to 50,000 and a salt thereof for producing an ophthalmic composition for suppressing contact lens dryness is furthermore provided.

Since the ophthalmic composition according to the present embodiment can exhibiting the effect of the present invention more remarkably, it is preferable that the ophthalmic composition be an ophthalmic agent (including an ophthalmic agent that can be applied while contact lenses are worn). When the ophthalmic composition according to the present embodiment is an ophthalmic agent, the ophthalmic composition is not particularly limited as long as the usage and dosage thereof are usage and a dosage that produce the effect and little side effects, and, for example, in the cases of an adult (15 years old or older) and an infant who is 7 years old or older, a method using 1 to 3 drops, 1 to 2 drops, or 2 to 3 drops per once 2 to 4 times or 5 to 6 times per day by application can be exemplified.

The ophthalmic composition according to the present embodiment is contained in any container and provided. The container for storing the ophthalmic composition according to the present embodiment is not particularly limited, and may be made of, for example, glass or plastic. The container is preferably made of plastic. Examples of the plastic include polyethylene terephthalate (PET), polyarylate, polyethylene naphthalate, polycarbonates, polyethylene, polypropylene, polyimides, copolymers of monomers constituting these, and mixtures of two or more of these. The plastic is preferably polyethylene terephthalate. The container for storing the ophthalmic composition according to the present embodiment may be a transparent container, the inside of which can be confirmed visibly, or an opaque container, the inside of which can be difficultly confirmed visibly. The container is preferably a transparent container. The “transparent container” used here includes both colorless, transparent containers and colored transparent containers.

A nozzle may be installed in the container for storing the ophthalmic composition according to the present embodiment. The material of the nozzle is not particularly limited, and the nozzle may be made of glass or plastic. The nozzle is preferably made of plastic. Examples of the plastic include polybutylene terephthalate, polyethylene, polypropylene, polyethylene terephthalate, polyethylene naphthalate, copolymers of monomers constituting these, and mixtures of two or more of these. As the material of the nozzle, polypropylene, polyethylene, polyethylene terephthalate, polybutylene terephthalate, and polyethylene naphthalate are preferable, and polyethylene is more preferable from the viewpoint of enhancing the effect of the invention further.

The container for storing the ophthalmic composition according to the present embodiment may be a multi-dose type, which stores a plurality of used amounts, or a unit-dose type, which stores a single used amount.

It is preferable that the ophthalmic composition according to the present embodiment be filled into the container having a capacity of 4 to 30 mL, it is more preferable that the ophthalmic composition be filled into the container having a capacity of 5 to 20 mL, it is further preferable that the ophthalmic composition be filled into the container having a capacity of 6 to 16 mL, and it is further more preferable that the ophthalmic composition be filled into the container having a capacity of 10 to 15 mL. The ophthalmic composition may be filled into a container having a capacity of 0.1 to 3 mL, or the ophthalmic composition may be filled into a container having a capacity of 0.2 to 1 mL.

### EXAMPLES

Although the present invention will be described based on the Test Examples specifically hereinafter, the present invention is not limited to these. All the sodium chondroitin sulfate to be used in the following Test Examples is derived from sharks.

### Test Example 1: Dryness Stress Test Using Rabbits

Sodium chondroitin sulfate having a weight average molecular weight of around 20,000 (Maruha Nichiro Corporation; sodium chondroitin sulfate in Japanese Pharmaceutical Codex) or sodium chondroitin sulfate having a weight average molecular weight of around 40,000 (SEIKAGAKU CORPORATION; grade N-K) was dissolved in a physiological saline solution (Otsuka Pharmaceutical Factory, Inc.) to 0.5% w/v or 3% w/v separately to prepare test substances.

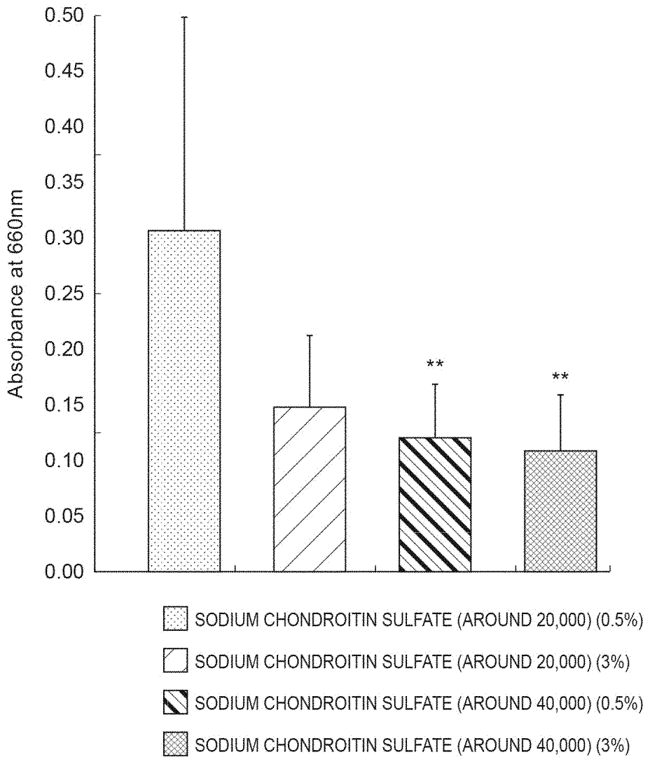

Rabbits (Japanese white rabbits (KITAYAMA LABES CO., LTD.)) classified into five groups (n=5) according to the method of Nagano et al. (Journal of the Eye, 13(2), 267-270, 1996) were put under general anesthesia, rings obtained by cutting finger portions of gloves into a ring form were then installed on the right and left eyeballs, and the eyelids were opened forcedly. Each test substance was applied to both eyes of each rabbit in each group in 100 μL per eye immediately after the eyelids were opened, and the rabbit was left to stand with the eyelids open for 3 hours. Then, 50 μL of 1% methylene blue solution was dropped on the eye surfaces, and the eye surfaces were stained and washed with physiological saline solution. The corneas were excised from the eye surfaces, methylene blue dye was then extracted, and the absorbance at 660 nm was measured using the microplate reader Multiskan GO (Thermo Fisher Scientific K.K.). The results are shown in FIG. 1. Since a cornea lesion is formed by eye dryness, and the amount of the dye attached to the cornea surfaces increases, it can be estimated that as the absorbance value increases, the eyes become dryer.

It was confirmed from FIG. 1 that the absorbance in the case of sodium chondroitin sulfate having a weight average molecular weight of around 40,000 was remarkably low as compared with sodium chondroitin sulfate having a weight average molecular weight of around 20,000 (**p<0.01, Tukey's multiple comparison test), and sodium chondroitin sulfate having a weight average molecular weight of around 40,000 suppressed a cornea lesion due to eye dryness.

### Test Example 2: Wettability of Contact Lenses

Ophthalmic compositions having the compositions shown in Table 1 were prepared in accordance with a usual method and used as Test Solutions. The unit of the components in Table 1 is % w/v. In the Test Example 2 and the following Test Examples, sodium chondroitin sulfate having a weight average molecular weight of around 20,000 (SEIKAGAKU CORPORATION; grade ND-K) and sodium chondroitin sulfate having a weight average molecular weight of around 40,000 (SEIKAGAKU CORPORATION; grade N-K) were used.

Then, 4 mL of a phosphate-buffered physiological saline solution (0.60% sodium chloride, 0.60% sodium hydrogen phosphate (dodecahydrate), 0.05% sodium dihydrogen phosphate (dihydrate), pH 7.4±0.1) was poured into each well in a 12-well plate (BD Falcon, No. 35-3043). One contact lens (ACUVUE OASYS (Johnson & Johnson K.K.)) was immersed in each well and left to stand at room temperature for 4 hours or more. Next, 4 mL of the phosphate-buffered physiological saline solution and 4 mL of each Test Solution were poured into each well in a 12-well plate. Water on the contact lens was lightly wiped off with lint-free paper, and the contact lens was immersed and left to stand for 15 minutes. Subsequently, 100 mL of the phosphate-buffered physiological saline solution was poured into a beaker. The immersed contact lens was lightly rinsed with the phosphate-buffered physiological saline solution, and water was removed using lint-free paper. The contact lens was placed on a slide glass, and the contact angle 0.10 seconds after 3 μl of the phosphate-buffered physiological saline solution was dropped was measured using a contact angle measuring device (solid-liquid interface analysis system DropMaster 500 (Kyowa Interface Science Co., Ltd.)). The results are shown in Table 1. It can be estimated that as contact angle decreases, the wettability of the contact lens becomes better, and the affinity for the contact lens increases.

TABLE 1

| | Test | Test | Test |
|---|---|---|---|
| | Solution 1 | Solution 2 | Solution 3 |
| Boric acid | 1.7 | 1.7 | 1.7 |
| Borax | 0.2 | 0.2 | 0.2 |
| Sodium chondroitin sulfate | — | 0.5 | — |
| (around 20,000) | | | |
| Sodium chondroitin sulfate | — | — | 0.5 |
| (around 40,000) | | | |
| Purified water | Balance | Balance | Balance |
| Total volume | 100 mL | 100 mL | 100 mL |
| Contact angle (°) | 113.4 | 118.1 | 78.2 |

The Test Solution 2, containing sodium chondroitin sulfate having a weight average molecular weight of around 20,000, exhibited a contact angle almost equivalent to that of the Test Solution 1, not containing sodium chondroitin sulfate. Meanwhile, it was confirmed that the contact angle of Test Solution 3, containing sodium chondroitin sulfate having a weight average molecular weight of around 40,000, was remarkably low, the wettability was remarkably improved, and the affinity for the contact lens was high as compared with Test Solution 2, containing sodium chondroitin sulfate having a weight average molecular weight of around 20,000.

When Test Solution 3-1, having the same composition as Test Solution 3 except that the content of sodium chondroitin sulfate (weight average molecular weight: around 40,000) of Test Solution 3 was adjusted to 1% w/v, and Test Solution 3-2, obtained by adding 50,000 units of retinol palmitate, 0.35% w/v polyoxyethylene hydrogenated castor oil 60, and 0.2% w/v polyoxyethylene castor oil 10 to Test Solution 3-1, were measured for the contact angle)(° by the same method as Test Example 2, the contact angle of Test Solution 3-1 was 67.4°, and the contact angle of Test Solution 3-2 was 44.2°.

### Test Example 3: Contact Lens Dryness Test

First, 4 mL of a phosphate-buffered physiological saline solution (0.60% sodium chloride, 0.60% sodium hydrogen phosphate (dodecahydrate), 0.05% sodium dihydrogen phosphate (dihydrate), pH 7.4±0.1) was poured into each well in a 12-well plate (BD Falcon, No. 35-3043). Two contact lenses (ACUVUE OASYS (Johnson & Johnson K.K.)) were immersed in each well and left to stand at room temperature for 4 hours or more. Water on the contact lenses was lightly wiped off with lint-free paper, the contact lenses were then immersed in a 12-well plate in which 4 mL of the phosphate-buffered physiological saline solution and 4 mL of each Test Solution were poured into each well and left to stand at room temperature for 24 hours. Water on the contact lenses was lightly wiped off with lint-free paper, the contact lenses were immersed in a 12-well plate in which 2 mL of cobalt (II) chloride colorimetric stock solution (FUJIFILM Wako Pure Chemical Corporation, distributer code: 031-19041) was poured into each well and shaken under the conditions of room temperature and 200 rpm for 5 minutes. Water on the contact lenses was lightly wiped off with lint-free paper, and the coloration of the soft contact lenses was observed immediately (0 minutes) after the contact lenses were placed on cover glasses and 5 minutes, 15 minutes, and 30 minutes after the contact lenses were placed on the cover glasses. When, in the present Test Example, the contact lenses are dried, the contact lenses are colored blue. The results obtained by estimating whether the contact lenses were colored or not according to the following criterion are shown in

Table 2.

A: Not colored

B: Slightly colored (less than 30% of the whole contact lens surface)

C: Clearly colored (30% or more of the whole contact lens surface)

TABLE 2

| | | Test | Test | Test |
|---|---|---|---|---|
| | | Solution 1 | Solution 2 | Solution 3 |
| 0 | minutes | A | A | A |
| 5 | minutes | A | A | A |
| 15 | minutes | B | B | A |
| 30 | minutes | C | C | B |

In the cases of Test Solution 1, not containing sodium chondroitin sulfate, and Test Solution 2, containing sodium chondroitin sulfate having a weight average molecular weight of around 20,000, slight coloration was observed 15 minutes after, and in the case of Test Solution 3, containing sodium chondroitin sulfate having a weight average molecular weight of around 40,000, the contact lenses were not meanwhile colored 15 minutes after, and slight coloration was observed 30 minutes after. It was therefore confirmed that sodium chondroitin sulfate having a weight average molecular weight of around 40,000 suppressed contact lens dryness as compared with sodium chondroitin sulfate having a weight average molecular weight of around 20,000.

### Test Example 4: Test on Wettability of Contact Lenses

Ophthalmic compositions having the compositions shown in Table 3 were prepared according to the usual method and used as Test Solutions. The unit of the components in Table 3 is % w/v unless otherwise specified in the table. In Test Example 4 and the following Test Examples, retinol palmitate having 1,740,000 IU/g was used. Water on the surfaces of contact lenses (ACUVUE ADVANCE (Johnson & Johnson K.K.)) immersed in 2 mL of a physiological saline solution for 4 hours or more was fully wiped off with lint-free paper, a droplet (around 1 μl) of each of the prepared ophthalmic compositions was then dropped, and the contact angle (static contact angle) to each contact lens 0.1 seconds after the dropping was measured using an automatic contact angle meter (solid-fluid interface analysis system DropMaster, DM-A501 (Kyowa Interface Science Co., Ltd.)). Each of the ophthalmic compositions was measured for the contact angle 3 times, and the average value thereof was calculated and defined as the contact angle of each of the ophthalmic compositions. The results are shown in Table 3. It can be estimated that as the contact angle decreases, the wettability of the contact lens becomes better, and the affinity for the contact lens increases.

TABLE 3

| | Test | Test | Test | Test | Test | Test | Test |
|---|---|---|---|---|---|---|---|
| Component name | Solution 4 | Solution 5 | Solution 6 | Solution 7 | Solution 8 | Solution 9 | Solution 10 |
| Sodium chondroitin sulfate | 0.5 | 0.5 | 0.5 | 0.5 | 0.5 | 0.5 | 0.5 |
| (around 40,000) | | | | | | | |
| Neostigmine methylsulfate | — | — | 0.005 | — | — | — | — |
| Allantoin | — | — | — | 0.3 | — | — | — |
| Dipotassium glycyrrhizinate | — | — | — | — | 0.25 | | — |
| Pyridoxine hydrochloride | — | — | — | — | — | 0.1 | — |
| Panthenol | — | — | — | — | — | — | 0.1 |
| d-α-Tocopherol acetate | — | — | — | — | — | — | — |
| Retinol palmitate | — | — | — | — | — | — | — |
| Potassium L-aspartate | — | — | — | — | — | — | — |
| Taurine | — | — | — | — | — | — | — |
| Hydroxypropyl methylcellulose | — | — | — | — | — | — | — |
| Boric acid | 1.2 | 1.2 | 1.2 | 1.2 | 1.2 | 1.2 | 1.2 |
| Borax | 0.1 | 0.1 | 0.1 | 0.1 | 0.1 | 0.1 | 0.1 |
| Polyoxyethylene hydrogenated | — | 0.35 | — | — | — | — | — |
| castor oil (HCO-60) | | | | | | | |
| Polyoxyethylene castor oil | — | 0.2 | — | — | — | — | — |
| (CO-10) | | | | | | | |
| Hydrochloric acid | q.s. | q.s. | q.s. | q.s. | q.s. | q.s. | q.s. |
| Purified water | Balance | Balance | Balance | Balance | Balance | Balance | Balance |
| Total volume | 100 mL | 100 mL | 100 mL | 100 mL | 100 mL | 100 mL | 100 mL |
| pH | 5.5 | 5.5 | 5.5 | 5.5 | 5.5 | 5.5 | 5.5 |
| Contact angle (°) | 85.5 | 73.7 | 73.5 | 76.5 | 81.0 | 79.9 | 76.5 |

| | Test | Test | Test | Test | Test |
|---|---|---|---|---|---|
| Component name | Solution 11 | Solution 12 | Solution 13 | Solution 14 | Solution 15 |
| Sodium chondroitin sulfate | 0.5 | 0.5 | 0.5 | 0.5 | 0.5 |
| (around 40,000) | | | | | |
| Neostigmine methylsulfate | — | — | — | — | — |
| Allantoin | — | — | — | — | — |
| Dipotassium glycyrrhizinate | — | — | | — | — |
| Pyridoxine hydrochloride | — | — | | — | — |
| Panthenol | — | — | | — | — |
| d-α-Tocopherol acetate | 0.05 | — | | — | — |
| Retinol palmitate | — | 25,000 units | — | — | — |
| Potassium L-aspartate | — | — | 1 | — | — |
| Taurine | — | — | — | 1 | — |
| Hydroxypropyl methylcellulose | — | — | — | — | 0.1 |
| Boric acid | 1.2 | 1.2 | 1.2 | 1.2 | 1.2 |
| Borax | 0.1 | 0.1 | 0.1 | 0.1 | 0.1 |
| Polyoxyethylene hydrogenated | 0.35 | 0.35 | — | — | — |
| castor oil (HCO-60) | | | | | |
| Polyoxyethylene castor oil | 0.2 | 0.2 | — | — | — |
| (CO-10) | | | | | |
| Hydrochloric acid | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| pH | 5.5 | 5.5 | 5.5 | 5.5 | 5.5 |
| Contact angle (°) | 67.5 | 48.0 | 38.3 | 62.3 | 78.8 |

The contact angles of Test Solutions 6 to 10 and 13 to 15 were remarkably low as compared with Test Solution 4, and the wettability was remarkably improved. The contact angles of Test Solutions 11 and 12 were remarkably low as compared with Test Solution 5, and the wettability was remarkably improved. That is, it was confirmed that the contact angles of Test Solutions each containing sodium chondroitin sulfate having a weight average molecular weight of around 40,000, and neostigmine methylsulfate, allantoin, dipotassium glycyrrhizinate, pyridoxine hydrochloride, panthenol, d-α-tocopherol acetate, retinol palmitate, potassium L-aspartate, taurine, or hydroxypropyl methylcellulose were remarkably low, the wettability was remarkably improved, and the affinities for the contact lens were high as compared with Test Solution containing only sodium chondroitin sulfate having a weight average molecular weight of around 40,000.

### Test Example 5: Measurement of Contact Angle in Stainless Steel Tube

Ophthalmic compositions having compositions shown in Table 4 were prepared according to the usual method and used as Test Solutions. The unit of the components in Table 4 is % w/v. A droplet (around 1 μl) of each prepared ophthalmic composition was dropped on a metal plate made of stainless steel (ferrule cap Type CLF-B), and the contact angle (static contact angle) to the metal 0.1 seconds after the dropping was measured using an automatic contact angle meter (solid-fluid interface analysis system DropMaster, DM-A501 (Kyowa Interface Science Co., Ltd.)). Each of the ophthalmic compositions was measured for the contact angles 3 times, and the average value was calculated and defined as the contact angle of the each of the ophthalmic compositions. The results are shown in Table 4.

TABLE 4

| | Test | Test |
|---|---|---|
| Component name | Solution 4 | Solution 15 |
| Sodium chondroitin sulfate | 0.5 | 0.5 |
| (around 40,000) | | |
| Hydroxypropyl methylcellulose | — | 0.1 |
| Boric acid | 1.2 | 1.2 |
| Borax | 0.1 | 0.1 |
| Hydrochloric acid | q.s. | q.s. |
| Purified water | Balance | Balance |
| Total volume | 100 mL | 100 mL |
| pH | 5.5 | 5.5 |
| Contact angle (°) | 65.0 | 72.2 |

Test Solution 15 remarkably increased in the contact angle as compared with Test Solution 4. That is, it was confirmed that Test Solution containing sodium chondroitin sulfate having a weight average molecular weight of around 40,000 and hydroxypropyl methylcellulose has a remarkably low affinity for a stainless steel tube as compared with Test Solution containing only sodium chondroitin sulfate having a weight average molecular weight of around 40,000. It is found that since filling tubes in production lines comprise a metal such as stainless steel, droplets attaching to the tips of the filling tubes can be reduced at the time of filling containers with the ophthalmic composition, and the equalization of the filling amount is facilitated.

### Test Example 6: Test Related to Preservative Effect

Ophthalmic compositions having compositions shown in Table 5 were prepared according to the usual method and used as Test Solutions. The unit of the components in Table 5 is % w/v unless otherwise specified in the table. Staphylococcus aureus (ATCC6538) was inoculated on the surface of soybean casein digest slant medium and cultured at 33° C. for 24 hours. The cultured bacterial cells were collected with a platinum loop aseptically and floated on a suitable amount of sterilized physiological saline solution to prepare a bacterial suspension containing live bacteria at around 1×10⁷ CFU/mL. The viable count of the suspension was measured by culturing the bacteria separately. A 15-mL conical tube made of PET (Corning Incorporated) was then filled with 10 mL of each of the prepared ophthalmic compositions. Staphylococcus aureus bacterial liquid (suspended in a physiological saline solution) was inoculated into each of these ophthalmic compositions so that the viable count (final concentration) was around 10⁵ CFU/mL, and the mixture was well-stirred to prepare a sample. The sample containing the bacteria was stored under a light-shielding condition at 23° C. for 3 days. The concentration of the sample containing the bacteria was then adjusted to a concentration suitable for counting, 1 mL of the diluted sample was seeded on a 3M™ Petri Film™ rapid aerobic count plate (RAC plate), and after culture at 33° C. for 2 days, the viable count was determined by counting the number of colonies observed. The viable count immediately after the inoculation and the viable count in the sample after storage for 3 days were compared, and a decrement in the bacterial count was calculated as Log Reduction. Furthermore, it was determined based on the calculated Log Reduction according to the following evaluation criterion whether the ophthalmic composition had an enough preservative effect. The bacteria were cultured at 33° C. for 2 days for counting the initial bacterial count. The results are shown in Table 5.

### Evaluation Criterion

### Log Reduction<0.7: D

### 0.7≤Log Reduction<0.8: C

### 0.8≤Log Reduction<0.9: B

### 0.9≤Log Reduction: A

TABLE 5

| | Test | Test | Test | Test | Test | Test |
|---|---|---|---|---|---|---|
| Component name | Solution 16 | Solution 17 | Solution 7 | Solution 8 | Solution 12 | Solution 14 |
| Sodium chondroitin sulfate | 0.5 | 0.5 | — | — | — | — |
| (around 20,000) | | | | | | |
| Sodium chondroitin sulfate | — | — | 0.5 | 0.5 | 0.5 | 0.5 |
| (around 40,000) | | | | | | |
| Allantoin | — | — | 0.3 | — | — | — |
| Dipotassium glycyrrhizinate | — | — | — | 0.25 | — | — |
| Retinol palmitate | — | — | — | — | 25,000 units | — |
| Taurine | — | — | — | — | — | 1 |
| Boric acid | 1.2 | 1.2 | 1.2 | 1.2 | 1.2 | 1.2 |
| Borax | 0.1 | 0.1 | 0.1 | 0.1 | 0.1 | 0.1 |
| Polyoxyethylene hydrogenated | — | 0.35 | — | — | 0.35 | — |
| castor oil 60 | | | | | | |
| Polyoxyethylene castor oil 10 | — | 0.2 | — | — | 0.2 | — |
| Hydrochloric acid | q.s. | q.s. | q.s. | q.s. | q.s. | q.s. |
| Purified water | Balance | Balance | Balance | Balance | Balance | Balance |
| Total volume | 100 mL | 100 mL | 100 mL | 100 mL | 100 mL | 100 mL |
| pH | 5.5 | 5.5 | 5.5 | 5.5 | 5.5 | 5.5 |
| Evaluation criterion | D | D | A | A | A | A |

It was confirmed that the preservative effects in Test Solutions 7, 8, 12, and 14, containing sodium chondroitin sulfate having a weight average molecular weight of around 40,000, and allantoin, dipotassium glycyrrhizinate, retinol palmitate, or taurine were further improved as compared with Test Solutions 16 and 17, containing sodium chondroitin sulfate having a weight average molecular weight of around 20,000. That is, it was confirmed that the preservative effects of Test Solutions containing sodium chondroitin sulfate having a weight average molecular weight of around 40,000 were improved as compared with sodium chondroitin sulfate having a weight average molecular weight of around 20,000, and the preservative effect was further improved by further incorporating allantoin, dipotassium glycyrrhizinate, retinol palmitate or taurine.

### Test Example 7: Viscosity Measurement

Ophthalmic compositions having compositions shown in Table 6 were prepared according to the usual method and used as Test Solutions. The unit of the components in Table 6 is % w/v unless otherwise specified in the table. The viscosity of each of the ophthalmic compositions (600 μl) after the preparation was measured for the viscosity at a shear rate at 34° C. with a rheometer (MCR302 (manufactured by Anton Paar Japan K.K.)) using a cone plate type jig (CP50-1, d: 0.102 mm) As to the viscosity (m·Pas) at a shear rate of 10,000 (1/s), the decrease rate of the viscosity of Test Solution 12 or 13 to Test Solution 4 or 5 (preparation in which sodium chondroitin sulfate alone was blended) was calculated according to the following expression. The viscosity decrease shows that the viscosity decreases when stress is applied, and the viscosity change at the time of palpebration can be evaluated. The viscosity decrease at the time of palpebration therefore indicates that palpebration is easy, and an uncomfortable feeling at the time of palpebration is hardly felt. The shear rate of 10000 (1/s) is assumed to be palpebration speed.

Viscosity decrease rate (%)=(1−viscosity of Test Solution 12 or Test Solution 13/viscosity of corresponding Test Solution)×100 (Expression)

The Test Solution corresponding to Test Solution 12 is Test Solution 5, and the Test Solution corresponding to Test Solution 13 is Test Solution 4.

The results are shown in Table 6.

TABLE 6

| | Test | Test | Test | | Test |
|---|---|---|---|---|---|
| Component name | Solution 4 | Solution 5 | Solution 12 | | Solution 13 |
| Sodium chondroitin sulfate | 0.5 | 0.5 | 0.5 | | 0.5 |
| (around 40,000) | | | | | |
| Retinol palmitate | — | — | 25,000 | units | — |
| Potassium L-aspartate | — | — | — | | 1 |
| Boric acid | 1.2 | 1.2 | 1.2 | | 1.2 |
| Borax | 0.1 | 0.1 | 0.1 | | 0.1 |
| Polyoxyethylene hydrogenated | — | 0.35 | 0.35 | | — |
| castor oil 60 | | | | | |
| Polyoxyethylene castor oil 10 | — | 0.2 | 0.2 | | — |
| Hydrochloric acid | q.s. | q.s. | q.s. | | q.s. |
| Purified water | Balance | Balance | Balance | | Balance |
| Total volume | 100 mL | 100 mL | 100 | mL | 100 mL |

| | | | | |
|---|---|---|---|---|
| pH | 5.5 | 5.5 | 5.5 | 5.5 |
| Viscosity decrease rate (%) | — | — | 17.4 | 29.9 |

The viscosity remarkably decreased in Test Solutions 12 and 13, containing retinol palmitate or potassium L-aspartate as compared with Test Solutions 5 and 4. Here, it was confirmed by the present inventors that the viscosity of Test Solution 4, using sodium chondroitin sulfate having a weight average molecular weight of around 40,000, measured with a rotational viscometer after the preparation was higher than the viscosity of Test Solution having the same composition as Test Solution 4 except that sodium chondroitin sulfate having a weight average molecular weight of around 20,000 was substituted for sodium chondroitin sulfate having a weight average molecular weight of around 40,000 in Test Solution 4. If the viscosity of the ophthalmic composition is high at the time of application, problems such as blinking difficultly and easily feeling discomfort may occur. It can therefore be said that if the viscosity at a high shear rate is low, blinking is easy after the application, and an uncomfortable feeling is hardly felt. It was therefore confirmed that the ophthalmic composition containing sodium chondroitin sulfate having a weight average molecular weight of around 40,000 and retinol palmitate or potassium L-aspartate facilitates blinking after the application, and prevents discomfort from being felt easily as compared with the ophthalmic composition containing only sodium chondroitin sulfate having a weight average molecular weight of around 40,000.

### Test Example 8: Viscosity Stability Test by Photoirradiation

Ophthalmic compositions having compositions shown in Table 7 was prepared according to the usual method and used as Test Solutions. The unit of the components in Table 7 is % w/v unless otherwise specified in the table. A glass headspace vial (GL Sciences Inc.) having a capacity of 10 mL was filled with 10 mL of each of the ophthalmic compositions, and the ophthalmic composition was irradiated with a photostability testing device (LT-120A-WCD (manufactured by NAGANO SCIENCE CO., LTD.)) using a D65 fluorescent lamp as a light source at a room temperature of 25° C. and an illumination of 4,000 1×/h until the accumulated illumination was 1,200,000 1×. Each of the ophthalmic compositions (600 μl) before and after the irradiation was measured for the viscosity at a shear rate (1 to 10,000 (1/s)) and 34° C. with a rheometer (MCR302 (Anton Paar Japan K.K.)) using a cone plate type jig (CP50-1, d: 0.102 mm) The viscosity stabilities before and after the test were evaluated according to the following expression using the viscosities (m·Pas) at a shear rate of 1,000 (1/s). The results are shown in Table 7. It is shown that as the viscosity change rate decreases, the viscosity change by light becomes slighter, and the physical properties of the ophthalmic composition are preserved in the same way as before.

Viscosity change rate (%)={(Viscosity of each of the ophthalmic compositions before photoirradiation−viscosity of each of the ophthalmic compositions after photoirradiation)/viscosity of each of the ophthalmic compositions before photoirradiation}×100 (Expression)

TABLE 7

| | Test | Test | Test | Test | Test |
|---|---|---|---|---|---|
| Component name | Solution 16 | Solution 17 | Solution 4 | Solution 5 | Solution 6 |
| Sodium chondroitin sulfate | 0.5 | 0.5 | — | — | — |
| (around 20,000) | | | | | |
| Sodium chondroitin sulfate | — | — | 0.5 | 0.5 | 0.5 |
| (around 40,000) | | | | | |
| Neostigmine methylsulfate | — | — | — | — | 0.005 |
| d-α-Tocopherol acetate | — | — | — | — | — |
| Retinol palmitate | — | — | — | — | — |
| Potassium L-aspartate | — | — | — | — | — |
| Taurine | — | — | — | — | — |
| Boric acid | 1.2 | 1.2 | 1.2 | 1.2 | 1.2 |
| Borax | 0.1 | 0.1 | 0.1 | 0.1 | 0.1 |
| Polyoxyethylene hydrogenated | — | 0.35 | — | 0.35 | — |
| castor oil 60 | | | | | |
| Polyoxyethylene castor oil 10 | — | 0.2 | — | 0.2 | — |
| Hydrochloric acid | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| Viscosity change rate at | 0.4 | 5.8 | 26.5 | 23.9 | 0.8 |
| shear rate of 1,000 (1/s) (%) | | | | | |

| | Test | Test | Test | Test |
|---|---|---|---|---|
| Component name | Solution 11 | Solution 12 | Solution 13 | Solution 14 |
| Sodium chondroitin sulfate | — | — | — | — |
| (around 20,000) | | | | |
| Sodium chondroitin sulfate | 0.5 | 0.5 | 0.5 | 0.5 |
| (around 40,000) | | | | |
| Neostigmine methylsulfate | — | — | — | — |
| d-α-Tocopherol acetate | 0.05 | — | — | — |
| Retinol palmitate | — | 25,000 units | — | — |
| Potassium L-aspartate | — | — | 1 | — |
| Taurine | — | — | — | 1 |
| Boric acid | 1.2 | 1.2 | 1.2 | 1.2 |
| Borax | 0.1 | 0.1 | 0.1 | 0.1 |
| Polyoxyethylene hydrogenated | 0.35 | 0.35 | — | — |
| castor oil 60 | | | | |
| Polyoxyethylene castor oil 10 | 0.2 | 0.2 | — | — |
| Hydrochloric acid | q.s. | q.s. | q.s. | q.s. |
| Purified water | Balance | Balance | Balance | Balance |
| Total volume | 100 mL | 100 mL | 100 mL | 100 mL |
| Viscosity change rate at | 2.6 | 4.3 | 1.4 | 0.5 |
| shear rate of 1,000 (1/s) (%) | | | | |

A problem that although the viscosity does not change by photoirradiation in Test Solutions 16 and 17, containing sodium chondroitin sulfate having a weight average molecular weight of around 20,000, the viscosity changes in Test Solutions 4 and 5, containing sodium chondroitin sulfate having a weight average molecular weight of around 40,000 by photoirradiation was found. It was however confirmed that the viscosity change rate decreased remarkably in Test Solution 6 and 11 to 14, containing sodium chondroitin sulfate having a weight average molecular weight of around 40,000 and neostigmine methyl sulfate, d-α-tocopherol acetate, retinol palmitate, potassium L-aspartate, or taurine, and the stability of the ophthalmic composition by photoirradiation was improved. When the viscosity was measured at a shear rate of 100 (1/s), a similar tendency was obtained.

### Test Example 9: Deposition Suppression Test

Ophthalmic compositions having compositions shown in Table 8 were prepared according to the usual method and used as Test Solutions. The unit of the components in Table 8 is % w/v. Then, 1 mL of each of the ophthalmic compositions was added to each well in a 24-well plate (Corning Incorporated) and stored in a dry heat dryer (IKEDA SCIENTIFIC Co., Ltd.) at 60° C. for 2 days, the state of deposition in each well in the plate was then visually observed, and the deposit generation was evaluated according to the following evaluation criterion. The results are shown in Table 8.

### <Evaluation Criterion of Deposit Generation>

A deposit that can be definitely confirmed is present on the undersurface of a well, and the rate of the deposit occupying the undersurface is ½ or more: +++
A deposit that can be definitely confirmed is present on the undersurface of a well, and the rate of the deposit occupying the undersurface is ⅓ or more and less than ½: ++
A deposit that can be definitely confirmed is present on the undersurface of a well, and the rate of the deposit occupying the undersurface is less than ⅓: +
A deposit is not present: −

TABLE 8

| | Test | Test |
|---|---|---|
| Component name | Solution 18 | Solution 13 |
| Sodium chondroitin sulfate | 0.5 | — |
| (around 20,000) | | |
| Sodium chondroitin sulfate | — | 0.5 |
| (around 40,000) | | |
| Potassium L-aspartate | 1 | 1 |
| Boric acid | 1.2 | 1.2 |
| Borax | 0.1 | 0.1 |
| Hydrochloric acid | q.s. | q.s. |
| Purified water | Balance | Balance |
| Total volume | 100 mL | 100 mL |
| Deposition | +++ | + |

The occurrence of deposition was remarkably suppressed in Test Solution 13, containing sodium chondroitin sulfate having a weight average molecular weight of around 40,000 and potassium aspartate as compared with Test Solution 18, containing sodium chondroitin sulfate having a weight average molecular weight of around 20,000 and potassium aspartate.

### Test Example 10: Appearance (Color) Change Suppression Test by Photoirradiation

Ophthalmic compositions having compositions shown in Table 9 were prepared according to the usual method and used as Test Solutions. The unit of the components in Table 9 is % w/v unless otherwise specified in the table. A glass headspace vial having a capacity of 10 mL was filled with 10 mL of each of the ophthalmic compositions, and the ophthalmic composition was irradiated with a photostability testing device (LT-120A-WCD (manufactured by NAGANO SCIENCE CO., LTD.)) using a D65 fluorescent lamp as a light source at a room temperature of 25° C. and an illumination of 4,000 1×/h until the accumulated illumination was 1,200,000 1×. After the test, each of the ophthalmic compositions before and after the photoirradiation was measured for a color difference change (b* value) with a spectrocolorimeter (CM3500d: manufactured by KONICA MINOLTA, INC.), a change in the appearance (color) of the ophthalmic composition (color difference change variation; Δb* value) by photoirradiation was calculated according to the following expression 1, and the decrease rate of the color difference change was further calculated according to the following expression 2. The results are shown in Table 9. It is indicated that as the Δb* value decreases, a change in the appearance (color) of the ophthalmic composition (coloration) is further suppressed.

Δb*=b*value of each of the ophthalmic compositions before photoirradiation−b*value of each of the ophthalmic compositions after photoirradiation (Expression 1)

Decrease rate of color difference change (%)={1−(Δb* of Test Solution 12/Δb* of Test Solution 19)}×100 (Expression 2)

TABLE 9

| | Test | | Test | |
|---|---|---|---|---|
| Component name | Solution 19 | | Solution 12 | |
| Sodium chondroitin sulfate | 0.5 | | — | |
| (around 20,000) | | | | |
| Sodium chondroitin sulfate | — | | 0.5 | |
| (around 40,000) | | | | |
| Retinol palmitate | 25,000 | units | 25,000 | units |
| Boric acid | 1.2 | | 1.2 | |
| Borax | 0.1 | | 0.1 | |
| Polyoxyethylene hydrogenated | 0.35 | | 0.35 | |
| castor oil 60 | | | | |
| Polyoxyethylene castor oil 10 | 0.2 | | 0.2 | |
| Hydrochloric acid | q.s. | | q.s. | |
| Purified water | Balance | | Balance | |
| Total volume | 100 | mL | 100 | mL |

| | | |
|---|---|---|
| pH | 5.5 | 5.5 |
| Decrease rate of color | — | 32.3 |
| difference change (%) | | |

It was confirmed that Test Solution 12, containing sodium chondroitin sulfate having a weight average molecular weight of around 40,000 and retinol palmitate has a small color difference change variation by photoirradiation (Δb*) as compared with Test Solution 19, containing sodium chondroitin sulfate having a weight average molecular weight of around 20,000 and retinol palmitate, and the coloration by photoirradiation was suppressed. Differences in the coloration degree between Test Solution 19 before and after the photoirradiation and between Test Solution 19 and Test Solution 12 after the photoirradiation were also visually observed.

### Test Example 11: Appearance (Color) Change Suppression Test by Ultraviolet Irradiation

Ophthalmic compositions having compositions shown in Table 10 were prepared according to the usual method and used as Test Solutions. The unit of the components in Table 10 is % w/v unless otherwise specified in the table. Then, 10 mL of each of the ophthalmic compositions was filled into a glass bottle (10 mL) and irradiated at 35° C. using an SUNTESTER XLS+ (manufactured by TOYO SEIKI CO., LTD., 1,700 W xenon air-cooling lamp light source) at an ultraviolet ray illumination of 765 (W/m²) for 96 hours. Then, each of the ophthalmic compositions was fully maintained at a constant temperature of 25° C., the color difference changes (b* value) of each of the ophthalmic compositions before and after the ultraviolet irradiation were measured using a spectrocolorimeter (CM3500d: manufactured by KONICA MINOLTA, INC.), a change in the appearance (color) of the ophthalmic composition before and after the ultraviolet irradiation (color difference change variation; Δb* value) was calculated according to the following expression 1, and the decrease rate of color difference change was further calculated according to the following expression 2. The results are shown in Table 9. It is indicated that as the Δb* value decreases, a change in the appearance (color) of the ophthalmic composition (coloration) is further is suppressed.

Δb*=b*value of each of the ophthalmic compositions before photoirradiation−b*value of each of the ophthalmic compositions after photoirradiation (Expression 1)

Decrease rate of color difference change (%)={1−(Δb* of Test Solution 12/Δb* of Test Solution 19)}×100 (Expression 2)

The Test Solution corresponding to Test Solution 9 is Test Solution 20 and the Test Solution corresponding to Test Solution 12 is Test Solution 19.

TABLE 10

| | Test | Test | | Test | Test | |
|---|---|---|---|---|---|---|
| Component name | Solution 20 | Solution 19 | | Solution 9 | Solution 12 | |
| Sodium chondroitin sulfate | 0.5 | 0.5 | | — | — | |
| (around 20,000) | | | | | | |
| Sodium chondroitin sulfate | — | — | | 0.5 | 0.5 | |
| (around 40,000) | | | | | | |
| Pyridoxine hydrochloride | 0.1 | — | | 0.1 | — | |
| Retinol palmitate | — | 25,000 | units | — | 25,000 | units |
| Boric acid | 1.2 | 1.2 | | 1.2 | 1.2 | |
| Borax | 0.1 | 0.1 | | 0.1 | 0.1 | |
| Polyoxyethylene hydrogenated | — | 0.35 | | — | 0.35 | |
| castor oil 60 | | | | | | |
| Polyoxyethylene castor oil 10 | — | 0.2 | | — | 0.2 | |
| Hydrochloric acid | q.s. | q.s. | | q.s. | q.s. | |
| Purified water | Balance | Balance | | Balance | Balance | |
| Total volume | 100 mL | 100 | mL | 100 mL | 100 | mL |

| | | | | |
|---|---|---|---|---|
| pH | 5.5 | 5.5 | 5.5 | 5.5 |
| Decrease rate of color | — | — | 23.0 | 38.7 |
| difference change (%) | | | | |

It was confirmed that Test Solutions 9 and 12, containing sodium chondroitin sulfate having a weight average molecular weight of around 40,000 and pyridoxine hydrochloride or retinol palmitate, had a small color difference change variation by the ultraviolet irradiation (Δb*), coloration by ultraviolet irradiation was suppressed as compared with Test Solutions 20 and 19, containing sodium chondroitin sulfate having a weight average molecular weight of around 20,000 and pyridoxine hydrochloride or retinol palmitate.

### Test Example 12: Appearance (Transparency) Change Suppression Test by Heat

Ophthalmic compositions having compositions shown in Table 11 were prepared according to the usual method and used as Test Solutions. The unit of the components in Table 11 is % w/v unless otherwise specified in the table. Then, 10 mL of each of the ophthalmic compositions was filled into a glass headspace vial having a capacity of 10 mL, and left to stand and kept in a thermostatic storage at 60° C. for 3 weeks (heat acceleration test). Then, each of the ophthalmic compositions was fully maintained at a constant temperature of 25° C., the color difference change (L* value) of each of the ophthalmic compositions before and after the heat acceleration test was measured using a spectrocolorimeter (CM3500d: manufactured by KONICA MINOLTA, INC.), a change in the appearance (transparency) (ΔL*) of the ophthalmic composition before and after the heat acceleration test was calculated according to the following expression 1, and the decrease rate of the transparency difference change was further calculated according to the following expression 2. The results are shown in Table 11. The L* value is used as an index indicating transparency. It is therefore indicated that as the ΔL* value decreases, a change in the appearance (transparency) of the preparation is further suppressed.

ΔL*=L*value before heat acceleration test−L*value after heat acceleration test (Expression 1)

Decrease rate of color difference change (%)={1−(ΔL* of Test Solution 11 or Test Solution 12/ΔL* of corresponding Test Solution}×100 (Expression 2)

Test Solution corresponding to Test Solution 11 is Test Solution 21, and Test Solution corresponding to Test Solution 12 is Test Solution 19.

TABLE 11

| | Test | Test | | Test | Test | |
|---|---|---|---|---|---|---|
| Component name | Solution 21 | Solution 19 | | Solution 11 | Solution 12 | |
| Sodium chondroitin sulfate | 0.5 | 0.5 | | — | — | |
| (around 20,000) | | | | | | |
| Sodium chondroitin sulfate | — | — | | 0.5 | 0.5 | |
| (around 40,000) | | | | | | |
| d-α-Tocopherol acetate | 0.05 | — | | 0.05 | — | |
| Retinol palmitate | — | 25,000 | units | — | 25,000 | units |
| Boric acid | 1.2 | 1.2 | | 1.2 | 1.2 | |
| Borax | 0.1 | 0.1 | | 0.1 | 0.1 | |
| Polyoxyethylene hydrogenated | 0.35 | 0.35 | | 0.35 | 0.35 | |
| castor oil 60 | | | | | | |
| Polyoxyethylene castor oil 10 | 0.2 | 0.2 | | 0.2 | 0.2 | |
| Hydrochloric acid | q.s. | q.s. | | q.s. | q.s. | |
| Purified water | Balance | Balance | | Balance | Balance | |
| Total volume | 100 mL | 100 | mL | 100 mL | 100 | mL |

| | | | | |
|---|---|---|---|---|
| pH | 5.5 | 5.5 | 5.5 | 5.5 |
| Decrease rate of | — | — | 82.1 | 61.5 |
| transparency change (%) | | | | |

It was confirmed that Test Solutions 11 and 12, containing sodium chondroitin sulfate having a weight average molecular weight of around 40,000 and d-α-tocopherol acetate or retinol palmitate had a remarkably small transparency change by heat, and a change in the appearance (transparency) by heat was suppressed as compared with Test Solutions 21 and 19, containing sodium chondroitin sulfate having a weight average molecular weight of around 20,000 and d-α-tocopherol acetate or retinol palmitate.

### Test Example 13: Cell Dryness Test

Ophthalmic compositions having compositions shown in Table 12 were prepared according to the usual method and used as Test Solutions. The unit of the components in Table 12 is % w/v unless otherwise specified in the table. Then, 100 μL of HCE-T cells, which were a human corneal epithelium cell line, were seeded in each well in a 96-well plate (Corning Incorporated) at a concentration of 1×10⁵ cells/mL and cultured in a CO₂ incubator set at 37° C., a humidity of 90%, and a CO₂ concentration of 5% until the cells were confluent. As growth medium, DMEM/F12 (Thermo Fisher Scientific K.K.) to which FCS (DS Pharma) was added to 5%, DMSO (FUJIFILM Wako Pure Chemical Corporation) was added to 0.5%, recombinant human EGF (R&D Systems Inc.) was added to 10 ng/mL, and insulin solution human (Merck KGaA) was added to 5 μg/mL was used. When the cells were confluent for 2 to 4 days after, the growth medium was suction-removed from each well, and 50 μL of each of the ophthalmic compositions were added to each well, and the cells were incubated at 37° C. under the condition of 5% CO₂ for 15 minutes. The each of the ophthalmic compositions was suction-removed from each well, dryness stress was then applied by leaving the cells to stand in a clean bench for 20 minutes, and the viable cell count was then evaluated. Next, 10 μL of the cell count measurement reagent CellCountingKit-8 (DOJINDO LABORATORIES) was added to each well for every 100 μL of the culture medium, and after culture at 37° C. under the conditions of 5% CO₂ and a humidity of 90% for 2 to 3 hours, measurement at 450 nm was performed using an absorbance meter (Molecular Devices, LLC) to evaluate the viable cell count. It is indicated that as the value of the absorbance increases, the viable cell count increases. The results are shown in Table 12.

TABLE 12

| | Test | Test | Test | Test | Test | Test | Test | Test |
|---|---|---|---|---|---|---|---|---|
| Component name | Solution 4 | Solution 5 | Solution 8 | Solution 11 | Solution 12 | Solution 13 | Solution 14 | Solution 22 |
| Sodium chondroitin sulfate | 0.5 | 0.5 | 0.5 | 0.5 | 0.5 | 0.5 | 0.5 | 1 |
| (around 40,000) | | | | | | | | |
| Dipotassium glycyrrhizinate | — | — | 0.25 | — | — | — | — | — |
| d-α-Tocopherol acetate | — | | | 0.05 | | | | |
| Retinol palmitate | — | — | — | — | 25,000 units | — | — | — |
| Potassium L-aspartate | — | — | — | — | — | 1 | — | — |
| Taurine | — | — | — | — | — | — | 1 | — |
| Boric acid | 1.2 | 1.2 | 1.2 | 1.2 | 1.2 | 1.2 | 1.2 | 1.2 |
| Borax | 0.1 | 0.1 | 0.1 | 0.1 | 0.1 | 0.1 | 0.1 | 0.1 |
| Polyoxyethylene hydrogenated | — | 0.35 | — | 0.35 | 0.35 | — | — | — |
| castor oil 60 | | | | | | | | |
| Polyoxyethylene castor oil 10 | — | 0.2 | — | 0.2 | 0.2 | — | — | — |
| Hydrochloric acid | q.s. | q.s. | q.s. | q.s. | q.s. | q.s. | q.s. | q.s. |
| Purified water | Balance | Balance | Balance | Balance | Balance | Balance | Balance | Balance |
| Total volume | 100 mL | 100 mL | 100 mL | 100 mL | 100 mL | 100 mL | 100 mL | 100 mL |
| pH | 5.5 | 5.5 | 5.5 | 5.5 | 5.5 | 5.5 | 5.5 | 5.5 |
| Absorbance | 0.50 | 0.52 | 0.74 | 0.63 | 0.67 | 0.70 | 0.64 | 0.95 |

It was confirmed that, in Test Solutions 8, 13, and 14, containing sodium chondroitin sulfate and dipotassium glycyrrhizinate, magnesium aspartate, or taurine, the viable cell counts increased remarkably, and cell death due to dryness stress was remarkably suppressed as compared with Test Solution 4, containing sodium chondroitin sulfate. It was confirmed that, in Test Solutions 11 and 12, containing d-α-tocopherol acetate or retinol palmitate, the viable cell counts increased remarkably, and cell death due to dryness stress was remarkably suppressed as compared with Test Solution 5, containing sodium chondroitin sulfate. Furthermore, it was confirmed from the results of Test Solutions 4 and 22 that even 1% sodium chondroitin sulfate increased the viable cell count remarkably.

### Test Example 14: Cell Damage Test

Ophthalmic compositions having compositions shown in Table 13 were prepared according to the usual method and used as Test Solutions. The unit of the components in Table 13 is % w/v. Then, 500 μL of HCE-T cells, which were a human corneal epithelium cell line, were seeded in each well in a 24-well plate (Corning Incorporated) at a concentration of 1×10⁵ cells/mL and cultured in a CO₂ incubator set at 37° C. and a CO₂ concentration of 5%. As growth medium, DMEM/F12 (Thermo Fisher Scientific K.K.) to which FCS (DS Pharma) was added to 5%, DMSO (FUJIFILM Wako Pure Chemical Corporation) was added to 0.5%, recombinant human EGF (R&D Systems Inc.) was added to 10 ng/mL, and insulin solution human (Merck KGaA) was added to 5 μg/mL was used. When the cells were confluent for 2 to 4 days after, the growth medium was suction-removed from each well, and 50 μL of each of the ophthalmic compositions were added to each well, and the cells were incubated at 37° C. under the condition of 5% CO₂ for 15 minutes. Three or four glass beads (AS ONE CORPORATION) were placed in each well, the plate was shaken at 450 rpm for 1 minute using a microplate shaker (Heidolph Instruments GmbH & Co. KG). The supernatant and the glass beads were removed, and 500 μL of culture medium in which the cell count measurement reagent CellCountingKit-8 (DOJINDO LABORATORIES) and culture medium were mixed at 1:10 was added, and after culture in a CO₂ incubator for 2 hours, the absorbance at 450 nm was measured using an absorbance meter (Molecular Devices, LLC). The cells viability was calculated by the following expression.

Cells viability (%)=(absorbance in each prescription/absorbance of control)×100 (Expression)

The results are shown in Table 13.

TABLE 13

| | | Test | Test | Test | Test | Test | Test |
|---|---|---|---|---|---|---|---|
| Component name | Control | Solution 16 | Solution 4 | Solution 7 | Solution 8 | Solution 9 | Solution 10 |
| Sodium chondroitin sulfate | — | 0.5 | — | — | — | — | — |
| (around 20,000) | | | | | | | |
| Sodium chondroitin sulfate | — | — | 0.5 | 0.5 | 0.5 | 0.5 | 0.5 |
| (around 40,000) | | | | | | | |
| Allantoin | — | — | — | 0.3 | — | — | — |
| Dipotassium glycyrrhizinate | — | — | — | — | 0.25 | — | — |
| Pyridoxine hydrochloride | — | — | — | — | — | 0.1 | — |
| Panthenol | — | — | — | — | — | — | 0.1 |
| Boric acid | 1.2 | 1.2 | 1.2 | 1.2 | 1.2 | 1.2 | 1.2 |
| Borax | 0.1 | 0.1 | 0.1 | 0.1 | 0.1 | 0.1 | 0.1 |
| Hydrochloric acid | q.s. | q.s. | q.s. | q.s. | q.s. | q.s. | q.s. |
| Purified water | Balance | Balance | Balance | Balance | Balance | Balance | Balance |
| Total volume | 100 mL | 100 mL | 100 mL | 100 mL | 100 mL | 100 mL | 100 mL |
| pH | 5.5 | 5.5 | 5.5 | 5.5 | 5.5 | 5.5 | 5.5 |
| Viability based on | — | 96.0 | 113.7 | 277.4 | 159.5 | 133.4 | 122.3 |
| control of 100 (%) | | | | | | | |

It was confirmed that, as to Test Solution 4, containing sodium chondroitin sulfate having a weight average molecular weight of around 40,000, the cell viability was high as compared with Test Solution 16, containing sodium chondroitin sulfate having a weight average molecular weight of around 20,000. It was confirmed that, as to the Test Solutions 4, 7, and 8 to 10, containing sodium chondroitin sulfate having a weight average molecular weight of around 40,000 and allantoin, dipotassium glycyrrhizinate, pyridoxine hydrochloride, or panthenol, the cell viability was remarkably high as compared with Test Solution 4. When external stimulus is given to the eyes (for example, when the eyes are rubbed with the hand, when blinks are conducted, when contact lenses are put in or removed, and when the eyes are subjected to friction against contact lenses and the invasion of foreign bodies (pollen, air pollutants, eyelashes, eye makeup, and other foreign bodies)) after the ophthalmic composition containing sodium chondroitin sulfate having a weight average molecular weight of around 40,000 and allantoin, dipotassium glycyrrhizinate, pyridoxine hydrochloride or the panthenol is applied, it is therefore expected that ocular cell damage is suppressed.

### Test 15: Measurement Test of Amount of Liquid Remaining in Eyedropper

Ophthalmic compositions having compositions shown in Table 14 were prepared according to the usual method and used as Test Solutions. The unit of the components in Table 14 is % w/v. The tare weight of an eyedropper made of PET and having a capacity of 10 mL was weighed, and 5 mL of each of the ophthalmic compositions was filled. The weight of each eyedropper after the each of the filled ophthalmic compositions was completely emptied was next measured, and the remaining liquid amount (g) and the improvement rate of the remaining liquid as compared with Test Solution 22(%) were calculated according to the following expressions 1 and 2.

Remaining liquid amount (g)=weight of eyedropper after ophthalmic composition is completely emptied−tare weight of eyedropper (Expression 1)

Improvement rate of remaining liquid amount (%)={1−(remaining liquid amount of each Test Solution/remaining liquid amount of Test Solution 22)}×100 (Expression 2)

TABLE 14

| | Test | Test | Test | Test | Test | Test | Test |
|---|---|---|---|---|---|---|---|
| Component name | Solution 22 | Solution 4 | Solution 5 | Solution 6 | Solution 11 | Solution 12 | Solution 14 |
| Sodium chondroitin sulfate | — | 0.5 | 0.5 | 0.5 | 0.5 | 0.5 | 0.5 |
| (around 40,000) | | | | | | | |
| Neostigmine methylsulfate | — | — | — | 0.005 | — | — | — |
| Taurine | — | — | — | — | — | — | 1 |
| d-α-Tocopherol acetate | — | — | — | — | 0.05 | — | — |
| Retinol palmitate | — | — | — | — | — | 25,000 units | — |
| Boric acid | 1.2 | 1.2 | 1.2 | 1.2 | 1.2 | 1.2 | 1.2 |
| Borax | 0.1 | 0.1 | 0.1 | 0.1 | 0.1 | 0.1 | 0.1 |
| Polyoxyethylene hydrogenated | — | — | 0.35 | — | 0.35 | 0.35 | — |
| castor oil (HCO-60) | | | | | | | |
| Polyoxyethylene castor oil | — | — | 0.2 | — | 0.2 | 0.2 | — |
| (CO-10) | | | | | | | |
| Hydrochloric acid | q.s. | q.s. | q.s. | q.s. | q.s. | q.s. | q.s. |
| Purified water | Balance | Balance | Balance | Balance | Balance | Balance | Balance |
| pH | 5.5 | 5.5 | 5.5 | 5.5 | 5.5 | 5.5 | 5.5 |
| Total volume | 100 mL | 100 mL | 100 mL | 100 mL | 100 mL | 100 mL | 100 mL |
| Improvement rate of | — | 23.6 | 32.2 | 27.6 | 34.1 | 37.7 | 25.8 |
| remaining liquid amount (%) | | | | | | | |

Although, in Test Solution 4 and 5, containing sodium chondroitin sulfate having a weight average molecular weight of around 40,000, the viscosity increases, the remaining liquid amount decreased as compared with Test Solution 22, not containing sodium chondroitin sulfate having a weight average molecular weight of around 40,000. It was confirmed that in Test Solutions 6 and 14, containing neostigmine methylsulfate or taurine in Test Solution 4 and Test Solutions 11 and 12, containing d-α-tocopherol acetate or retinol palmitate in Test Solution 5, the remaining liquid amount decreased remarkably, and the ophthalmic compositions in the eyedroppers were easily used up.

### Preparation Examples

Ophthalmic agents are prepared by the usual method using the prescription according to following Tables 15 to 17. The unit of the component amounts in following Tables 15 to 17 is % w/v unless otherwise specified in the tables.

TABLE 15

| | Preparation | Preparation | Preparation | Preparation | Preparation |
|---|---|---|---|---|---|
| Component name | Example 1 | Example 2 | Example 3 | Example 4 | Example 5 |
| Sodium chondroitin sulfate | 0.5 | 0.5 | 3.0 | 0.5 | 3.0 |
| Tetrahydrozoline hydrochloride | 0.01 | — | — | — | — |
| Neostigmine methylsulfate | — | 0.001 | — | — | — |
| Epsilon-aminocaproic acid | — | — | — | — | 0.5 |
| Allantoin | — | — | 0.3 | — | — |
| Dipotassium glycyrrhizinate | 0.25 | — | — | — | — |
| Chlorpheniramine maleate | 0.03 | 0.02 | — | — | 0.03 |
| Flavin adenine dinucleotide | — | — | — | — | — |
| sodium | | | | | |
| Retinol palmitate | — | 35,000 units | — | — | 50,000 units |
| Pyridoxine hydrochloride | 0.1 | 0.05 | 0.05 | — | — |
| d-α-Tocopherol acetate | — | 0.035 | — | — | 0.005 |
| Potassium L-aspartate | — | — | — | — | — |
| Magnesium potassium L-aspartate | — | — | — | — | — |
| Taurine | — | 1 | 0.5 | — | — |
| Potassium chloride | — | — | — | 0.08 | — |
| Sodium chloride | — | — | — | 0.45 | — |
| l-Menthol | 0.01 | — | 0.04 | 0.015 | 0.2 |
| d-Camphor | 0.005 | — | 0.01 | — | — |
| d-Borneol | 0.003 | — | — | 0.015 | — |
| Geraniol | — | 0.005 | 0.003 | — | — |
| Eucalyptus oil | — | — | — | — | 0.001 |
| Bergamot oil | — | — | — | — | 0.001 |
| Mint oil | — | 0.001 | — | — | — |
| Polyoxyethylene hydrogenated | — | 0.2 | — | — | 0.5 |
| castor oil | | | | | |
| Polyoxyethylene castor oil | 0.1 | — | — | 0.3 | — |
| Polysorbate 80 | 0.5 | — | 0.3 | — | 0.2 |
| Polyoxyl stearate | — | — | — | — | — |
| Polyoxyethylene | — | 0.1 | — | 0.1 | — |
| polyoxypropylene glycol | | | | | |
| Benzalkonium chloride | — | — | 0.01 | — | — |
| Benzethonium chloride | — | — | — | — | 0.01 |
| Chlorhexidine gluconate | — | — | — | — | — |
| Polyhexanide hydrochloride | — | — | — | — | — |
| Chlorobutanol | — | — | 0.15 | — | — |
| Zinc chloride | — | 0.00025 | — | — | — |
| Sodium edetate | 0.01 | 0.05 | — | — | 0.05 |
| Boric acid | 1.5 | 1.8 | 1 | — | 1.5 |
| Borax | 0.5 | 0.35 | — | — | 0.3 |
| Sodium hydrogen phosphate | — | — | — | — | — |
| Sodium dihydrogen phosphate | — | — | — | — | — |
| Citric acid | — | — | — | — | — |
| Sesame oil | — | — | — | 0.1 | — |
| Propylene glycol | — | — | — | — | — |
| Concentrated glycerin | — | — | — | 0.1 | — |
| Sodium hyaluronate | — | — | 0.1 | — | 0.02 |
| L-Arginine | — | — | — | — | — |
| Polyvinylpyrrolidone | — | — | — | — | — |
| Hydroxyethyl cellulose | — | — | — | 0.25 | — |
| Hypromellose | — | — | — | 0.25 | — |
| Carboxyvinyl polymer | — | — | — | — | — |
| Dibutylhydroxytoluene | — | — | — | — | 0.01 |
| Hydrochloric acid | q.s. | q.s. | q.s. | q.s. | q.s. |
| Sodium hydroxide | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| pH | 7.0 | 6.5 | 5.7 | 7.2 | 6.9 |

| | Preparation | Preparation | Preparation | Preparation | Preparation |
|---|---|---|---|---|---|
| Component name | Example 6 | Example 7 | Example 8 | Example 9 | Example 10 |
| Sodium chondroitin sulfate | 0.3 | 1 | 0.1 | 0.5 | 1 |
| Tetrahydrozoline hydrochloride | — | — | — | 0.05 | — |
| Neostigmine methylsulfate | — | — | — | 0.005 | — |
| Epsilon-aminocaproic acid | — | — | — | 1 | 2 |
| Allantoin | — | — | — | — | 0.1 |
| Dipotassium glycyrrhizinate | — | — | — | 0.1 | 0.15 |
| Chlorpheniramine maleate | — | — | — | — | 0.03 |
| Flavin adenine dinucleotide | — | — | — | — | 0.05 |
| sodium | | | | | |
| Retinol palmitate | — | — | — | 50,000 units | — |
| Pyridoxine hydrochloride | — | — | 0.1 | 0.05 | 0.1 |
| d-α-Tocopherol acetate | — | — | 0.03 | — | 0.05 |
| Potassium L-aspartate | — | 1000 | — | 1 | — |
| Magnesium potassium L-aspartate | — | — | 0.2 | — | — |
| Taurine | 0.5 | — | 0.5 | 1 | — |
| Potassium chloride | 0.08 | 0.1 | — | — | — |
| Sodium chloride | 0.4 | 0.3 | — | — | — |
| l-Menthol | 0.008 | 0.05 | 0.002 | 0.002 | — |
| d-Camphor | — | — | — | 0.003 | — |
| d-Borneol | — | — | — | — | — |
| Geraniol | — | 0.002 | — | — | — |
| Eucalyptus oil | — | — | 0.002 | — | — |
| Bergamot oil | — | — | — | — | — |
| Mint oil | — | — | 0.002 | — | — |
| Polyoxyethylene hydrogenated | 0.3 | — | — | 0.1 | — |
| castor oil | | | | | |
| Polyoxyethylene castor oil | 0.1 | — | — | — | — |
| Polysorbate 80 | — | 0.3 | 0.5 | — | — |
| Polyoxyl stearate | 0.05 | — | — | — | — |
| Polyoxyethylene | — | 0.05 | — | — | — |
| polyoxypropylene glycol | | | | | |
| Benzalkonium chloride | — | — | 0.01 | 0.01 | — |
| Benzethonium chloride | — | — | — | — | — |
| Chlorhexidine gluconate | — | 0.02 | — | — | — |
| Polyhexanide hydrochloride | 1 ppm | — | — | — | 1.5 ppm |
| Chlorobutanol | — | — | — | — | 0.3 |
| Zinc chloride | — | — | — | — | — |
| Sodium edetate | — | 0.01 | 0.01 | 0.05 | 0.05 |
| Boric acid | 1 | — | 1 | 0.4 | 1 |
| Borax | 0.2 | — | 0.1 | 0.1 | 0.2 |
| Sodium hydrogen phosphate | — | 0.15 | — | — | — |
| Sodium dihydrogen phosphate | — | 0.2 | — | — | — |
| Citric acid | — | — | — | 0.1 | — |
| Sesame oil | 0.05 | 0.05 | — | — | — |
| Propylene glycol | 0.1 | — | — | — | — |
| Concentrated glycerin | — | — | — | — | — |
| Sodium hyaluronate | 0.3 | — | 0.1 | — | — |
| L-Arginine | — | — | 0.5 | — | — |
| Polyvinylpyrrolidone | — | — | — | 0.5 | — |
| Hydroxyethyl cellulose | 0.05 | 0.6 | — | — | 0.1 |
| Hypromellose | — | — | — | — | — |
| Carboxyvinyl polymer | — | 0.1 | — | — | — |
| Dibutylhydroxytoluene | — | — | — | 0.005 | — |
| Hydrochloric acid | q.s. | q.s. | q.s. | q.s. | q.s. |
| Sodium hydroxide | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| pH | 7.0 | 7.5 | 7.3 | 7.0 | 5.5 |

TABLE 16

| | Preparation | Preparation | Preparation | Preparation | Preparation |
|---|---|---|---|---|---|
| Component name | Example 11 | Example 12 | Example 13 | Example 14 | Example 15 |
| Sodium chondroitin | 0.5 | 1 | 0.1 | 0.25 | 0.1 |
| sulfate (around 40,000) | | | | | |
| Tetrahydrozoline hydrochloride | 0.01 | 0.03 | 0.1 | 0.01 | 0.01 |
| Neostigmine methylsulfate | — | 0.005 | — | 0.005 | 0.005 |
| Epsilon-aminocaproic acid | — | 1 | 1 | 1 | 1 |
| Allantoin | 0.1 | — | — | — | — |
| Dipotassium glycyrrhizinate | — | 0.1 | 0.25 | — | — |
| Sodium azulenesulfonate | — | — | — | — | — |
| Beiberine chloride | — | — | — | — | — |
| Zinc sulfate | — | 0.05 | — | — | — |
| Chlorpheniramine maleate | — | 0.03 | 0.03 | 0.03 | 0.03 |
| Flavin adenine dinucleotide | — | — | 0.05 | — | — |
| sodium | | | | | |
| Cyanocobalamin | — | 0.01 | — | — | — |
| Retinol palmitate | — | 50,000 units | — | 100,000 units | 50,000 units |
| Pyridoxine hydrochloride | — | 0.05 | 0.1 | — | 0.01 |
| d-α-Tocopherol acetate | 0.03 | — | — | 0.05 | 0.03 |
| Potassium L-aspartate | 0.5 | 0.5 | 0.5 | — | 0.8 |
| Magnesium potassium L-aspartate | — | — | — | 0.800 | — |
| Taurine | 0.5 | 0.5 | 1 | 0.5 | 1 |
| Potassium chloride | — | 0.008 | — | — | — |
| Sodium chloride | — | — | — | — | — |
| l-Menthol | 0.02 | 0.01 | — | 0.005 | 0.008 |
| d-Camphor | — | 0.001 | — | — | — |
| d-Borneol | — | 0.001 | — | 0.001 | — |
| Geraniol | 0.001 | — | 0.002 | — | — |
| Eucalyptus oil | 0.001 | — | — | 0.001 | — |
| Bergamot oil | — | — | — | 0.001 | — |
| Mint oil | — | — | — | — | 0.001 |
| Polyoxyethylene hydrogenated | — | — | 0.3 | 0.3 | 0.4 |
| castor oil | | | | | |
| Polyoxyethylene castor oil | — | — | — | — | — |
| Polysorbate 80 | 0.2 | — | — | 0.2 | 0.2 |
| Polyoxyl stearate | — | — | — | — | — |
| Polyoxyethylene | 0.05 | — | — | — | — |
| polyoxypropylene glycol | | | | | |
| Benzalkonium chloride | — | 0.01 | — | — | — |
| Chlorhexidine gluconate | — | — | — | — | — |
| Polyhexanide hydrochloride | 1 ppm | — | 1 ppm | — | — |
| Chlorobutanol | 0.1 | 0.2 | 0.1 | — | — |
| Zinc chloride | — | — | — | — | — |
| Sodium edetate | 0.01 | 0.05 | 0.01 | 0.05 | 0.05 |
| Boric acid | 1.2 | 0.2 | 0.3 | 0.1 | 0.5 |
| Borax | 0.3 | 0.1 | 0.05 | 0.025 | — |
| Sodium hydrogen phosphate | — | — | — | — | — |
| Sodium dihydrogen phosphate | — | — | — | — | — |
| Citric acid | — | — | — | — | — |
| Trometamol | — | — | — | — | 0.3 |
| Sesame oil | — | — | — | — | — |
| Propylene glycol | — | — | — | — | 0.2 |
| Concentrated glycerin | 0.1 | — | — | — | — |
| Sodium hyaluronate | 0.005 | 0.002 | — | — | — |
| Dextran | — | — | — | 0.05 | — |
| L-Arginine | — | — | — | — | — |
| Polyvinylpyrrolidone | — | — | — | — | — |
| Hydroxyethyl cellulose | — | — | 0.1 | — | — |
| Hydroxypropyl methylcellulose | 0.1 | — | — | — | — |
| Dibutylhydroxy toluene | — | — | — | 0.01 | 0.01 |
| Hydrochloric acid | q.s. | q.s. | q.s. | q.s. | q.s. |
| Sodium hydroxide | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| pH | 5.8 | 6.5 | 5.7 | 6.2 | 5.8 |

| | Preparation | Preparation | Preparation | Preparation | Preparation |
|---|---|---|---|---|---|
| Component name | Example 16 | Example 17 | Example 18 | Example 19 | Example 20 |
| Sodium chondroitin | 0.5 | 0.25 | 0.4 | 0.25 | 0.15 |
| sulfate (around 40,000) | | | | | |
| Tetrahydrozoline hydrochloride | 0.02 | — | — | — | 0.02 |
| Neostigmine methylsulfate | — | — | — | — | 0.0025 |
| Epsilon-aminocaproic acid | — | — | — | 1 | — |
| Allantoin | — | 0.1 | — | — | — |
| Dipotassium glycyrrhizinate | 0.25 | 0.1 | — | — | 0.25 |
| Sodium azulenesulfonate | — | — | 0.02 | — | — |
| Beiberine chloride | 0.01 | — | 0.01 | — | — |
| Zinc sulfate | — | 0.1 | — | — | 0.1 |
| Chlorpheniramine maleate | 0.02 | 0.01 | 0.03 | — | — |
| Flavin adenine dinucleotide | — | — | 0.01 | 0.05 | — |
| sodium | | | | | |
| Cyanocobalamin | — | — | — | — | — |
| Retinol palmitate | 10,000 units | — | 150,000 units | — | — |
| Pyridoxine hydrochloride | 0.08 | — | — | 0.01 | — |
| d-α-Tocopherol acetate | — | 0.025 | 0.01 | — | — |
| Potassium L-aspartate | — | — | 0.1 | 1 | — |
| Magnesium potassium L-aspartate | — | 1 | — | 2 | — |
| Taurine | — | 0.5 | 0.1 | 1 | — |
| Potassium chloride | — | — | — | — | — |
| Sodium chloride | — | 0.1 | — | — | — |
| l-Menthol | 0.001 | 0.005 | — | 0.0045 | — |
| d-Camphor | — | — | — | 0.001 | — |
| d-Borneol | — | — | — | — | — |
| Geraniol | — | 0.0001 | 0.003 | — | — |
| Eucalyptus oil | — | — | 0.003 | — | — |
| Bergamot oil | 0.0005 | — | — | — | — |
| Mint oil | 0.0005 | — | — | — | 0.001 |
| Polyoxyethylene hydrogenated | — | — | — | 0.2 | — |
| castor oil | | | | | |
| Polyoxyethylene castor oil | — | 0.1 | — | — | — |
| Polysorbate 80 | — | 0.3 | 0.5 | 0.1 | 0.25 |
| Polyoxyl stearate | — | 0.1 | — | — | — |
| Polyoxyethylene | — | — | — | — | — |
| polyoxypropylene glycol | | | | | |
| Benzalkonium chloride | — | — | — | — | — |
| Chlorhexidine gluconate | — | — | — | 0.005 | — |
| Polyhexanide hydrochloride | — | — | — | — | |
| Chlorobutanol | — | 0.2 | — | — | — |
| Zinc chloride | — | — | — | — | 0.001 |
| Sodium edetate | — | 0.01 | 0.01 | 0.05 | 0.05 |
| Boric acid | 0.3 | 0.1 | 1 | 0.4 | 0.1 |
| Borax | — | 0.01 | 0.1 | 0.1 | 0.2 |
| Sodium hydrogen phosphate | — | — | — | — | 0.15 |
| Sodium dihydrogen phosphate | — | — | — | — | 0.2 |
| Citric acid | — | — | — | 0.1 | — |
| Trometamol | — | — | — | — | — |
| Sesame oil | — | 0.001 | — | — | — |
| Propylene glycol | — | — | — | — | — |
| Concentrated glycerin | — | — | — | — | — |
| Sodium hyaluronate | — | — | — | — | 0.01 |
| Dextran | — | — | — | — | — |
| L-Arginine | — | — | — | — | 0.1 |
| Polyvinylpyrrolidone | — | 0.1 | — | — | — |
| Hydroxyethyl cellulose | — | — | — | — | — |
| Hydroxypropyl methylcellulose | — | 0.2 | — | 0.01 | — |
| Dibutylhydroxy toluene | — | — | — | — | — |
| Hydrochloric acid | q.s. | q.s. | q.s. | q.s. | q.s. |
| Sodium hydroxide | q.s. | q.s. | q.s. | q.s. | q.s. |
| Purified water | Balance | Balance | Balance | Balance | Balance |
| Total volume | 100 mL | 100 mL | 100 mL | 100 mL | 100 mL |
| pH | 7 | 5.3 | 6 | 7 | 5.5 |

TABLE 17

| | Preparation | Preparation | Preparation | Preparation | Preparation | Preparation |
|---|---|---|---|---|---|---|
| Component name | Example 21 | Example 22 | Example 23 | Example 24 | Example 25 | Example 26 |
| Sodium chondroitin sulfate | 0.2 | 1 | 0.5 | 0.3 | 0.5 | 0.5 |
| (around 40,000) | | | | | | |
| Potassium L-aspartate | — | — | 1 | — | 0.5 | 0.5 |
| Magnesium potassium L-aspartate | — | 1 | — | — | 0.5 | — |
| Taurine | — | 0.5 | — | 1 | — | 0.5 |
| Potassium chloride | — | — | 0.08 | — | — | 0.05 |
| Sodium chloride | 0.6 | 0.4 | 0.1 | 0.3 | 0.45 | 0.4 |
| l-Menthol | 0.04 | 0.002 | 0.005 | 0.015 | — | 0.005 |
| d-Camphor | 0.01 | — | — | 0.003 | — | 0.003 |
| Geraniol | 0.003 | — | — | — | — | — |
| Eucalyptus oil | — | 0.002 | — | — | — | — |
| Bergamot oil | — | — | — | 0.001 | — | — |
| Mint oil | — | 0.002 | — | — | — | — |
| Polyoxyethylene hydrogenated | — | — | 0.1 | 0.1 | — | — |
| castor oil | | | | | | |
| Polyoxyethylene castor oil | — | — | — | — | 0.3 | — |
| Polysorbate 80 | 0.3 | 0.2 | — | — | — | 0.2 |
| Polyoxyl stearate | — | — | — | — | 0.1 | — |
| Polyoxyethylene | — | — | — | — | 0.05 | — |
| polyoxypropylene glycol | | | | | | |
| Benzalkonium chloride | — | 0.01 | 0.01 | — | — | — |
| Chlorhexidine gluconate | — | — | — | — | 0.02 | — |
| Polyhexanide hydrochloride | 1 ppm | — | — | 1 ppm | — | — |
| Chlorobutanol | 0.15 | — | — | — | — | — |
| Zinc chloride | — | — | — | — | — | 0.0001 |
| Sodium edetate | — | 0.005 | 0.05 | 0.005 | 0.01 | 0.05 |
| Boric acid | 1.5 | 1. | 0.4 | 1. | — | 1 |
| Borax | — | 0.1 | 0.1 | 0.2 | — | 0.2 |
| Sodium hydrogen phosphate | — | — | — | — | 0.15 | — |
| Sodium dihydrogen phosphate | — | — | — | — | 0.1 | — |
| Citric acid | — | — | 0.1 | — | — | 0.1 |
| Sesame oil | — | — | — | — | 0.05 | — |
| Concentrated glycerin | — | — | — | — | — | 0.1 |
| Sodium hyaluronate | — | 0.02 | — | | | |
| L-Arginine | — | 0.5 | — | — | — | — |
| Polyvinylpyrrolidone | — | — | 0.5 | 0.1 | — | — |
| Hydroxyethyl cellulose | 0.07 | — | — | — | 0.2 | — |
| Hydroxypropyl methylcellulose | — | — | 0.1 | — | 0.2 | 0.3 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | Balance | Balance | Balance | Balance | Balance | Balance |
| Total volume | 100 mL | 100 mL | 100 mL | 100 mL | 100 mL | 100 mL |
| pH | 6.5 | 7.2 | 6.8 | 7 | 7.5 | 6.2 |

1. An ophthalmic composition for suppressing eye dryness, comprising:
at least one selected from the group consisting of chondroitin sulfate having a weight average molecular weight of 30,000 to 50,000 and a salt thereof.

2. An ophthalmic composition for suppressing contact lens dryness, comprising:
at least one selected from the group consisting of chondroitin sulfate having a weight average molecular weight of 30,000 to 50,000 and a salt thereof.

3. An ophthalmic composition, comprising:
at least one selected from the group consisting of chondroitin sulfate having a weight average molecular weight of 30,000 to 50,000 and a salt thereof; and
at least one selected from the group consisting of an anti-inflammatory agent, vitamin A, vitamin B, vitamin E, aminoethylsulfonic acid and a salt thereof, aspartic acid and a salt thereof, neostigmine and a salt thereof, and a cellulosic polymer compound.

4. The ophthalmic composition according to claim 3, wherein the anti-inflammatory agent is at least one selected from the group consisting of allantoin and a salt thereof and glycyrrhizinic acid and a salt thereof, the vitamin B is at least one selected from the group consisting of panthenol and pyridoxine and a salt thereof, and the cellulosic polymer compound is at least one selected from the group consisting of hydroxypropyl methylcellulose and a salt thereof.
